(19) United States
(12) Patent Application Publication
(10) Pub. No.: US 2024/0299578 A1
(43) Pub. Date: Sep. 12, 2024

(54) An Oral Insulin Delivery Salt Nano-Precipitate

(71) Applicant: MONASH UNIVERSITY MAYALSIA, Selangor (MY)

(72) Inventor: Md Ezharul Hoque CHOWDHURY, Kuala Lumpur (MY)

(21) Appl. No.: 18/546,142

(22) PCT Filed: Jan. 24, 2022
(86) PCT No.: PCT/MY2022/050004

§ 371 Date: Aug. 11, 2023

(30) Foreign Application Priority Data
Feb. 15, 2021 (MY) PI2021000801
Jan. 24, 2022 (WO) PCT/MY2022/050004

(51) Int. Cl. A61K 47/69; A61K 9/00; A61K 33/00; A61K 38/28; A61P 3/10

(52) U.S. Cl. CPC A61K 47/6923; A61K 9/0053; A61K 33/00; A61K 38/28; A61K 47/6929; A61P 3/10

(57) ABSTRACT

An oral delivery salt nano-precipitate for oral delivery of protein therapeutics such as insulin. The salt nano-precipitate comprises a cation-providing inorganic metal salt and an anion-providing salt. The protein molecule is bound to the salt nano-precipitate by way of electrostatic interactions. Both the cation-providing and anion-providing salts are present in the nano-precipitate at a sufficiently high concentration to enable adequate saturation of cationic and anionic salts, per volume of the nano-precipitate, such that the target protein molecule is strongly bound therewith and buffered against premature release at an acidic pH.

This invention relates to an oral drug delivery agent. More particularly, this invention relates to an oral drug delivery agent for protein therapeutics.

### DESCRIPTION OF THE PRIOR ART

Protein therapeutics are increasingly becoming a critical part of pharmaceutical treatment for a growing number of diseases. Advancements in biotechnology and a better understanding of the pathophysiology of clinical conditions has led to the recognition of a growing number of proteins and peptides as potential therapeutics. There is currently a scarcity of non-invasive oral mode of administration for protein therapeutics. Proteins are naturally hydrophilic molecules with poor absorbance through the intestines and are prone to degradation by harsh pH and enzymatic action (Curto et. al. (2011))¹.

Perhaps, the first ever protein molecule used as a therapeutic is insulin, the cornerstone of treatment for type 1 and type 2 diabetes mellitus. The only therapeutically deliverable form of insulin presently available is injectable insulin. Regular and very frequent subcutaneous administrations are associated with low patient compliance and multiple injection site injuries. It is not currently possible to orally administer insulin as insulin is a peptide that is known to be not resistant to stomach acid or enzymatic degradation.

Oral drug administration is generally recognized as delivery of drugs through the gastrointestinal (GI) tract where the starting point for a drug is in the mouth or buccal cavity and absorption can take place at different portions of the small and large intestines (Zaman R. et. al. (2016))². The GI tract comprises several organs with each organ having its own pH and enzymatic environment. The stomach and small intestines have pH environments and secretary enzymes that specialize in modifying and breaking peptide and protein molecules and these organs can destroy any protein drug that passes through them non-discriminatorily (Allen C. et. al (2011))³.

In addition to pH and enzymes, another problem to be overcome is intestinal absorption of high molecular weight protein molecules in intact form. The intestinal wall is covered with a mucus layer, which is a combination of glycoproteins and bicarbonate ions. Beneath the mucus is the epithelium, the basement membrane (non-cellular layer) and a layer of submucosa that holds blood vessels and lymphatic ducts. There are different modes of uptake of molecules (phagocytosis, absorption through Peyer's patches or endocytosis by enterocytes) depending on the size, surface charge, hydrophilic or hydrophobic nature of the molecule (Banga A. K. (2006))⁴. Absorption of an intact protein generally fails due to its size and hydrophilic nature.

There have been a number of strategies proposed for an oral protein formulation with the most common approach being to attach a protein molecule to another molecule that envelops and creates a carrier for it. Nanoparticles are candidates for this.

U.S. Pat. No. 8,859,004 82 discloses a pH-sensitive insulin-loaded nanoparticle for oral insulin delivery. The nanoparticle comprises an enteric coating polymer (a pH-sensitive polymer), hydrophobic material, internal stabilizer, external stabilizer content and insulin. A modified double emulsion solvent evaporation method is used to prepare the nanoparticle.

U.S. Pat. No. 9,101,547 82 discloses an enteric-coated capsule containing insulin-loaded catonic nanoparticles for oral insulin delivery. The enteric-coated capsule encloses a plurality of nanoparticles and a solubilizer. Each of the nanoparticles comprises a polycationic polymer, a biodegradable polymer, insulin and a stabilizer.

Polymeric nanoparticles, such as poly(lactide-co-glycolide) (PLGA) and its derivatives have also been explored. Insulin incorporated into a blended polymer of polyfumanc anhydride (FA) and PLGA 50:50 (FA: PLGA) showed a reduction of glucose load in fasted rats for 3 hours (Mathiowitz E. et, al. (1997)⁵, Ensign L. M. et. al. (2012)⁶) Another formulation of HP55-coated capsule containing PLGA/RS nanoparticle-loaded insulin gradually lowered glucose levels for 10 to 15 hours (Wu Z. M. et. al. (2012)⁷).

Another oral nano-insulin delivery tool under consideration is chitosan and its derivatives. U.S. Pat. No. 9,828,445 81 discloses modified chitosan particles for delivering oral insulin. Chitosan particles are amidated with an amino acid or a fatty acid and subsequently grafted with N-isopropylacryamide and cross-linked to form modified chitosan particles. Insulin is then loaded onto the modified chitosan particles. pH-sensitive chitosan particles showed a 15-hour long effect of reducing blood glucose levels in diabetic rats, although, this started with a burst release (Pan Y. et. al. (2002)⁸). Similarly, TBA (thiolated polymer 2-iminothiolane)-attached chitosan with incorporated insulin showed glucose-lowering effect for 24 hours In non-diabetic rats (Krauland A. H. et. al. (2004)⁹).

However, due to the poor bioavailability of insulin following oral administration using such polymeric materials, the overall effect in lowering blood glucose level is undesirably limited.

Lysosome-entrapped insulin (LEI) are also being studied for oral administration of insulin (Molero J. E. et. al. (1982)¹⁰, Stefanov et. al. (1980)¹¹), although, none of the formulations qualified for clinical trial. One example of this is oral insulin 338(1338), which was created by a combination of amino acid substitution and linkage to a carrier molecule. The clinical trial for this oral insulin candidate was discontinued as oral insulin 338(1338) was found to be only effective at a very high dose due to limited bioavailability, which would make production costs unacceptably high (Halberg I. et, al. (2018)¹²).

Another oral insulin formulation candidate is insulin tregopil (Blocon), a human insulin analogue attached to a methoxy-triethyleneglycol-propionyl moiety linked to the Lys-β29 amino group and formulated with sodium caporate. In animal trials (dog), the bioavailability of this candidate was observed to be not very high (0.82% to 0.85%) (Drucker D. J. (2019)¹³, Gregory J. M. et. al. (2019)¹⁴).

ORMD-0801 from Oramed is currently in Phase 2 clinical trial and is prepared by attaching insulin to a permeation enhancer, soybean trypsin inhibitor and a chelator. Pre-clinical stage bioavailability in animal studies was estimated at 5 to 8% (Arbit E. et. al. (2017)¹⁵). Another oral insulin formulation under consideration is Diasome, a liver-targeting formulation. Current available data shows that oral dosing of Diasome is less effective compared to subcutaneous administration (Geho W. B. et, al. (2014)¹⁶). This is likely due to Diasome's lack of ability to overcome the multiple barriers of the gastrointestinal tract before being absorbed across the intestinal epithelium.

Precipitation-based salt particles have also been considered for insulin loading for oral delivery. One such example is strontium-substituted carbonate apatite which has shown only very limited success in terms of binding and releasing insulin in in vitro medium (Ahmad A. et. al. (2015)¹⁷). The low rate of insulin binding by the salt particles could be due to formation of a limited number of particles, whereas fast particle dissolution can also lead to unexpected premature insulin release from the degrading salt particles.

There remains a need for the development of an oral delivery agent for proteins and peptides that can overcome the multiple physico-chemical barriers of the GI tract while retaining sufficient bioavailability to generate an efficacious therapeutic response.

This invention thus aims to alleviate some or all of the problems of the prior art.

### SUMMARY OF THE INVENTION

In accordance with an aspect of the invention, there is provided an oral delivery salt nano-precipitate for oral delivery of a protein molecule. The nano-precipitate comprises an inorganic metal salt capable of conferring a cation-rich domain to the nano-precipitate for electrostatic binding with the negative charges of a target protein molecule and an anion-providing salt capable of conferring an anion-rich domain to the nano-precipitate for electrostatic binding with the positive charges of a target protein molecule. Both the cation-providing and anion-providing salts are present in the nano-precipitate at a sufficiently high concentration to enable adequate saturation of cationic and anionic salts, per volume of the nano-precipitate, such that the target protein molecule is strongly bound therewith and buffered against premature release at an acidic pH.

The cation-providing inorganic metal salt may be a Group 2 metal salt.

The ratio of the cation-providing inorganic metal salt to the anion-providing salt may be 5:2.

The cation-providing inorganic metal salt may be a barium salt (Ba²⁺), a strontium salt (Sr²⁺), a calcium salt (Ca²⁺), a ferrous salt (Fe²⁺) or a zinc salt (Zn²⁺).

The anion-providing salt may be a sulphate (SO₄²⁻).

The anion-providing salt may be a sulphite (SO₃²⁻).

The anion-providing salt may be a carbonate (CO₃²⁻).

The nano-precipitate may be barium sulphate (BaSO₄).

The nano-precipitate may be barium sulphite (BaSO₃).

The nano-precipitate may be barium carbonate (BaCO₃).

In a second aspect of the invention, there is provided a method of producing the salt nano-precipitate of the invention. The method comprises the following steps:
(i) providing a volume of the cation-providing inorganic metal salt;
(ii) providing a volume of the anion-providing salt; and
(iii) mixing the salts of steps (i) and (ii) to form a salt precipitate.

The cation-providing inorganic metal salt may be a Group 2 metal salt. The Group 2 metal salt may be barium chloride (BaCl₂).

The anion-providing salt may be sodium sulphate (Na₂SO₄).

The anion-providing salt may be sodium sulphite (Na₂SO₃).

The anion-providing salt may be sodium carbonate (Na₂CO₃).

Protein molecule loading may be performed by adding a volume of protein molecules to the cationic metal salt solution of step (i), before addition of the anionic salt solution of step (ii), and, mixing the constituents until a precipitate is formed.

The protein-loaded salt nano-precipitates may be surface-modified with a bio-adhesive protein. The bio-adhesive protein may be transferrin, casein or a folate binding protein.

The protein molecule loaded into the salt nano-precipitate may be insulin. The insulin-loaded salt nano-precipitate may be used in the treatment of hyperglycemia, type 1 or type 2 diabetes mellitus in an animal or a human subject. Such use may include oral administration of bicarbonate before treatment with the insulin-loaded salt nano-precipitates.

The present invention seeks to overcome the problems of the prior art by providing salt nano-precipitates having both cation (positive) and anion (negative) rich-domains conferred by an inorganic metal salt (cation-providing salt) and an anion providing salt. Due to the heterogeneous charge distribution, these salt nano-precipitates are capable of binding protein molecules through electrostatic interactions.

In particular, the salt nano-precipitates of this invention are very effective carriers of protein molecules that carry both positive and negative charges such as insulin, peptide hormones (e.g. GLP-1), enzymes, cytokines and monoclonal antibodies. These protein molecules have positive and negative charges because of the amine and carboxylic groups that interact, respectively, with the anion and cation rich domains of the salt nano-precipitates. In fact, every protein molecule has a net charge of either negative or positive at physiological pH, which enables the protein to easily bind to cation or anion rich domains of the salt nano-precipitates of this invention.

As explained in the previous section, the main technical barriers for oral delivery of protein molecules include rapid degradation of the protein molecule before arrival at the target site (due to pH ranging from basic to very acidic in the stomach and small intestine) as we as limited permeability of the intestinal lining for protein molecules (primarily due to the mucin barrier).

The salt nano-precipitates of this invention are able to overcome these barriers while retaining the efficacy of the protein molecule i.e. providing effcacious therapeutic bioavailability of the protein molecule in a subject. The cation and anion rich-domains of the salt nano-precipitates of this invention enable not only good protein loading but also resistance to premature release of the protein molecule before the target site. Presence of the cation-providing and anion-providing salts in the nano-precipitate at a sufficiently high concentration to enable adequate saturation of cationic and anionic salts, per volume of the nano-precipitate is key to the strong binding of the target protein molecule and effective buffering against premature release at an acidic pH. This enables the protein molecule to be protected against degradation by harsh pH and enzymatic environments.

The heterogenous charge nature of the salt nano-precipitates of this invention also enables it to efficiently bind to mucin i.e. enable the protein molecule bound to the salt nano-precipitate of this invention to effectively overcome the mucin barrier, be absorbed through the intestinal wall and enter systemic blood circulation so as to be bioavailable.

There is no harsh temperature or pH required during the precipitation of and/or protein loading into the salt nano-precipitates of this invention. This is a distinct advantage as protein molecules are prone to degradation or inactivation in harsh temperatures or pHs. The synthesis conditions of the salt nano-precipitates of this invention can be easily modifiable depending on the protein molecule and target site of delivery. Modifying the synthesis conditions can also influence protein-loading capacity and the protein-release profile of the salt nano-precipitate.

Various other advantages of the oral delivery salt nano-precipitate of this invention will be further elaborated in the following pages.

### BRIEF DESCRIPTION OF THE DRAWINGS

The invention is illustrated, although not limited, by the following description of embodiments made with reference to the accompanying drawings in which:

FIG. 1 is a simplified schematic representation of the precipitation reaction.

FIG. 2 is a simplified illustration of insulin loading into the salt nano-precipitates.

FIGS. 3A, 3B and 3C show FT-IR spectrums of barium sulphate (BaSO₄), barium sulphite (BSO₃) and barium carbonate (BaCO₃), respectively.

FIGS. 4A, 4B and 4C show Energy Dispersive X-Ray Spectroscopy (EDX) analysis of barium sulphate (BaSO₄), barium sulphite (BaSO₃) and barium carbonate (BaCO₃), respectively.

FIG. 5 show dissolution profiles of barium sulphate (BaSO₄), barium sulphite (BaSO₃) and barium carbonate (BaCO₃), upon exposure to pH 7.78 to pH 1.78 over a period of 3 hours.

FIG. 6A shows the percentage of loading of FITC-insulin into BaSO₄, BaSO₃ and BaCO₃.

FIG. 68 shows the percentage of release of FITC-insulin at lower pHs (pH 7.14 to pH 1.78).

FIGS. 7A, 7B and 7C are FE-SEM micrographs of BaSO₄, BaSO₃ and BaCO₃ particles, respectively.

FIGS. 8A, 8B, 8C, 8D and SE are FE-SEM images of free BaSO₄ particles, low concentration (1×) BaSO₄ particles loaded with 2 μg, 10 μg, 50 μg insulin, and, high concentration (20×) BaSO₄ particles loaded with insulin, respectively.

FIGS. 9A, 9B and 9C are FE-SEM images of free BaSO₃ particles, low concentration (1×) insulin-loaded BaSO₃ particles and high concentration (20×) insulin-loaded BaSO₃ particles, prepared with 50 μg of insulin, respectively.

FIGS. 10A, 10B and 10C are FE-SEM images of free BaCO₃ particles, low concentration (1×) insulin-loaded BaCO₃ particles and high concentration (20×) insulin-loaded BaCO₃ particles, respectively.

FIGS. 11A, 11B and 11C are FT-IR spectrums showing mucin adhesion to BaSO₃, BaSO₃ and BaCO₃ particles, respectively.

FIG. 12 shows mucin adhesion efficiency of BaSO₄, BaSO₃ and BaCO₃ particles.

FIG. 13 is a graph showing the effects of oral delivery of free insulin versus insulin-loaded BaSO₄, BaSO₃ and BaCO₃ particles to STZ-induced diabetic rats.

FIG. 14 are graphs showing the effect of oral administration of insulin-loaded BaSO₄, BaSO₃ and BaCO₃ particles to STZ-induced diabetic rats.

FIG. 15 is a graph showing the effect on blood glucose levels of pre-administering bicarbonate before oral gavage of insulin-loaded BaSO₄, BaSO₃ or BaCO₃ particles.

FIG. 16 is a graph showing the effect on blood glucose levels of oral administration (a) only with insulin, (b) only with insulin-loaded BaSO₄ particles, and (c) with bicarbonate followed by insulin-loaded BaSO₄ particles.

FIG. 17 show pictures of SDS-PAGE gels with different band intensities for albumin-bound BaSO₄, BaSO₃ and BaCO₃ particles following Coomassie staining,

FIG. 18 show the albumin band pictures of albumin-loaded BaSO₄, BaSO₃ and BaCO₃ particles following exposure to trypLE enzyme in medium with pH 7.4, 6.8 and 1.8.

FIG. 19 is a graph showing the relative percentage of albumin degradation calculated from the band intensities.

FIG. 20A show gel pictures of free albumin, followed by free albumin exposed to sGF and albumin-loaded BaSO₄ particles exposed to sGF.

FIG. 20B show gel pictures of free albumin, followed by free albumin exposed to sGF and albumin-loaded BaSO₃ particles exposed to sGF.

FIG. 20C show gel pictures of free albumin, followed by free albumin exposed to sGF and albumin-loaded BaCO₃ particles exposed to sGF.

FIG. 21 is a graph showing the effect on blood glucose levels over a 6-hour period following oral administration of SrSOrinsutin conjugation (200 IU/kg of insulin) after a 14-hour starvation.

FIG. 22 is a graph showing the effect on blood glucose levels over a 6-hour period following oral administration of SrSOr-nsulin conjugation (100 IU/kg of insulin) after a 14-hour starvation.

FIG. 23 is a graph showing the effect on blood glucose levels over a 6-hour period following oral administration of SrSOrinsulin conjugation (50 IU/kg of insulin) after a 14-hour starvation.

FIG. 24 is a graph showing the effect on blood glucose levels over a 6-hour period following oral administration of SrSOrinsulin conjugation (100 IU/kg of insulin) without starvation.

FIG. 25 is a graph showing the effect on blood glucose levels over a 6-hour period following oral administration of SrSO₃-insulin conjugation (100 IU/kg of insulin) after a 14-hour starvation.

FIG. 26 is a graph showing the effect on blood glucose levels over a 6-hour period following intravenous administration of free insulin.

FIG. 27 is a graph showing the effect on blood glucose levels over a 6-hour period following oral gavage of insulin-loaded transferrin modified BaSO₄.

FIG. 28 is a graph showing the effect on blood glucose levels over a 4-hour period following oral gavage of insulin-loaded casein modified BaSO₄.

FIG. 29 is a graph showing the percentage of reduction in blood glucose levels over a 4-hour period following oral gavage of insulin-loaded casein modified BaSO₄.

### DETAILED DESCRIPTION OF THE EMBODIMENTS

The present invention relates to an oral delivery salt nano-precipitate for oral delivery of protein molecules. The salt nano-precipitate of this invention mainly comprises a cation-providing inorganic metal salt and an anion-providing salt. The target protein molecule is bound to the nano-precipitate by way of electrostatic interactions.

### The Cation-Providing Inorganic Metal Salt

The cation-providing inorganic metal salt of this invention can be any suitable inorganic metal salt that is capable of conferring a cation-rich domain to the salt nano-precipitate for electrostatic binding with the negative charges of a target protein molecule.

The cation-providing inorganic metal salt may be any one of a barium salt (Ba²⁺), a strontium salt (Sr²⁺), a calcium salt (Ca²⁺), a ferrous salt (Fe²⁺) or a zinc salt (Zn²⁺). Preferably, the cation-providing inorganic metal salt is a Group 2 metal salt.

In a preferred embodiment, the cation-providing inorganic metal salt is a barium salt (Ba²⁺) that can be provided by way of any water-soluble barium salt such as barium chloride (BaCl₂).

The cation-providing inorganic metal salt must be present in the salt nano-precipitate of this invention at a sufficient concentration to confer a cation-rich domain to the salt nano-precipitate for electrostatic binding with the negative charges of a target protein molecule. The cation-providing inorganic metal salt should be present in the salt nano-precipitate of this invention at a concentration of between about 1 mM to about 1 M, preferably, between about 10 mM to about 1 M, and, most preferably, between about 100 mM to about 1 M.

### The Anion-Providing Salt

The anion-providing salt of this invention can be any suitable salt that is capable of conferring an anion-rich domain to the salt nano-precipitate for electrostatic binding with the positive charges of a target protein molecule.

The anion-providing salt may be any one of a sulphate (SO₄²⁻), a sulphite (SP₃²⁻) or a carbonate (CO₃²⁻) that can be provided by way or any water-soluble salt such as sodium sulphate (Na₂SO₄), sodium sulphite (Na₂SO₃), and sodium carbonate (Na₂CO₃).

The anion-providing salt must be present in the salt nano-precipitate of this invention at a sufficient concentration to confer an anion-rich domain to the salt nano-precipitate for electrostatic binding with the positive charges of a target protein molecule. The anion-providing salt should be present in the salt nano-precipitate of this invention at a concentration of between about 1 mM to about 1 M, preferably, between about 10 mM to about 1 M, and, most preferably, between about 100 mM to about 1 M,

### The Salt Nano-Precipitate

The salt nano-precipitate of this invention is built on a concept of complexing a target protein molecule with inorganic salt particles on the basis of ionic interactions. Due to the heterogeneous charge distribution, the salt nano-precipitates of this invention are capable of binding target therapeutic protein molecules like insulin through electrostatic interactions. For example, insulin (5.8 kDa) has localized surface charges (either positive or negative at basic or acidic pH). These surface charges of insulin allow it to electrostatically bind with either the cationic or anionic domains of the salt nano-precipitates, depending on the pH of the host solution.

The ratio of the cation-providing inorganic metal salt to the anion-providing salt in the salt nano-precipitate of this invention is preferably about 5:2. As demonstrated in the Examples at the end of the description, the inventors have surprisingly found that this ratio of cation-providing salt to anion-providing salt results in a high rate of particle formation. One possible explanation for this is that the cation-providing salts act as a powerful driving force to accelerate the precipitation reaction needed for particle formation.

For oral delivery of insulin, the inventors have surprisingly found that the preferred salt nano-precipitates of this invention are barium sulphate (BaSO₄), barium sulphite (BaSO₃) and barium carbonate (BaCO₃), with the most preferred compounds being barium sulphate (BaSO₄) and barium carbonate (BaCO₃).

From field emission scanning electron microscopy (FE-SEM) analysis of the preferred salt nano-precipitates of this invention i.e. BaSO₄, BaSO₃ and BaCO₃, the inventors observed the following regarding the morphology of the precipitates in terms of particle size, shape and aggregation pattern.

Surprisingly, the inventors noticed that there was a distinct morphological difference between the low salt concentration (1×) and high salt concentration (20×) salt nano-precipitates. This was true for all three Ba salt nano-precipitates of this invention i.e. BaSO₄, BaSO₃ and BaCO₃. Further details are provided in the Examples. Notably, it is postulated that the distinct morphology of the high concentration (20×) salt nano-precipitates is a pertinent factor in the improved efficacy of the high concentration salt nano-precipitates when compared to the low concentration salt nano-precipitates, in reducing blood glucose levels in test subjects (diabetic rats).

The low concentration (1×) BaSO₄ particles have a distinctive irregular, oval to hollow round shape. The larger particles have an oval to hollow round shape with a particle size of between 110 to 200 nm and the smaller particles are irregular shaped with a particle size of between 30 to 100 nm. On the other hand, for the high concentration (20×) BaSO₄ particle formulation, only smaller round shape particles were observed.

The low concentration (1×) BaSO₃ particles ranged in size from large (about 90 to 270 nm) to very small (about 15 to 40 nm) sized particles and were found to be of a round shape and held in tight baseball-shaped clusters with a rough surface area. Each distinctive particle cluster ranged from about 500 nm to 1.0 μm in diameter. Larger particles displayed a rounder shape with smooth surface area without forming clusters. The particles in the high concentration (20×) BaSO₃ formulation were relatively less aggregated with formation of clusters that look irregular, with no change in single particle size and shape.

The low concentration (1×) BaCO₃ particles were observed to have a distinctive square to rectangular shaped morphology, with the particle size varying widely from about 50 to 500 nm. The BaCO₃ particles in the low concentration formulation were also observed to be closely aggregated with one another and had a tendency to form rod or filament-like structures. In the high concentration (20×) formulation, the BaCO₃ particle clusters formed long, thin threads that appeared to be inter-tangled with each other, with no change in single particle morphology.

One of the biggest challenges in oral delivery of protein therapeutics is pre-systemic degradation due to fluctuating pH conditions, for example, extremely acidic (˜pH 1) pH in stomach when patient is in a fasting state. Hence, any oral delivery agent needs to be resistant to extreme acidic pH to survive transport through the gastrointestinal system.

From in vitro testing of the preferred salt nano-precipitates of BaSO₄, BaSO₃ and BaCO₃, the inventors observed that the BaSO₄ particles demonstrated an excellent resistance to degradation over a wide range of pH (from pH 1 to pH 7.78) over a 3-hour period. BaCO₃ also maintained almost the same level of resistance to degradation throughout the range of pH, with no significant particle loss at low pH. However, the inventors observed that BaSO₃ particles experienced significant degradation at pH 1.69.

Resistance to both basic and acidic pHs through a period of 3 hours (l stomach and intestinal residence time) demonstrates that the salt nano-precipitate of this invention can withstand the harsh gastrointestinal pH and enzymatic environment and protect the target protein molecule against pH and enzymatic degradation.

In addition to resistance to pH-induced degradation, an ability to efficiently bind with mucin is also an important indicator of an oral delivery agent's capacity for intestinal absorption. Mucins are highly O-glycosylated molecules that have gel-like properties and play an important role in protecting the intestines from luminal digestive enzymes, abrasion by food particles, and pathogens (i.e. chemical or physical injury) by forming a barrier between the lumen and the intestinal epithelium. In order to be absorbed into the intestinal lumen, oral delivery agents containing target protein therapeutics need to successfully pass through the mucin barrier.

Following in vitro tests (Fourier Transform Infrared Spectroscopy (FT-IR) and spectrophotometric protein quantification), the inventors observed that the preferred salt nano-precipitates of BaSO₄, BaSO₃ and BaCO₃ did indeed have an affinity to mucin and would therefore be capable of crossing the intestinal lining.

FT-IR analysis confirmed mucin binding by all of BaSO₄, BaSO₃, BaCO₃.

Muon-adhered BaSO₄ particles demonstrated 12 distinctive peaks (see FIG. 11A), with peaks 2360 cm⁻¹, 1188 cm⁻¹, 1064 cm⁻¹, 982 cm⁻¹, 635 cm⁻¹ and 605 cm⁻¹ matching the peaks of mucin-free BaSO₄ particles in similar positions. Peak 3415 cm⁻¹ showed a deviation to 2392 cm⁻¹ probably due to mucin attachment. Similarly, mucin peaks at 3279 cm⁻¹, 1549 cm⁻¹ and 1434 cm⁻¹ were observed to be shifted to 3275 cm⁻¹, 1539 cm⁻¹ and 1439 cm⁻¹, respectively, in mucin-adhered BaSO₄ particles. Existence of characteristic peaks for BaSO₄ and mucin indicates adherence or binding of BaSO₄ particles to mucin.

Mucin-adhered BaSO₃ particles showed 13 distinctive peaks (see FIG. 11B). The peaks of BaSO₃ particles, 1133 cm⁻¹, 912 cm⁻¹, 630 cm⁻¹ and 409 cm⁻¹ were found at 1131 cm⁻¹ 919 cm⁻¹, 509 cm⁻¹ and 474 cm⁻¹ positions in mucin-adhered BaSO₃ particles. On the other hand, 8 characteristic peaks from mucin were found at 3311 cm⁻¹, 1648 cm⁻¹, 1539 cm⁻¹, 1434 cm⁻¹, 1298 cm⁻¹, 1343 cm⁻¹, 1187 cm⁻¹ and 1101 cm⁻¹ positions. Mucin peaks showed deviation from their positions due to binding to BaSO₃ particles. The presence of characteristic peaks for BaSO₃ as well as for mucin indicates positive adhesion of BaSO₃ particles to mucin.

Mucin-adhered BaCO₃ particles demonstrated 9 distinctive peaks (see FIG. 11C). Mucin characteristic peaks at 3279 cm⁻¹, 1634 cm⁻¹, 1549 cm⁻¹, 1232 cm¹, 1115 cm⁻¹ were found respectively at 3384 cm⁻¹, 1645 cm⁻¹, 1555 cm⁻¹, 1213 cm⁻¹, 1121 cm⁻¹ positions in mucin-adhered BaCO₃ particles. The presence of characteristic peaks for BaCO₃ particles and mucin indicates positive adhesion of BaCO₃ particles to mucin.

The percentage of mucin adhesion to BaSO₄, BaSO₃, BaCO₃ was calculated by measuring the protein content (mucin) precipitated out with the particles after centrifugation. Following protein quantification by the Bradford method, the inventors observed that BaSO₄ particles showed the highest mucin adhesion (100%), followed by BaSO₃ and BaCO₃ particles with about 60 to 70% mucin adhesion. Such high mucin adhesion (i.e. the salt nano-precipitates of this invention has the ability to efficiently bind to mucin) facilitates the uptake of the salt nano-precipitates across the intestinal epithelium for intestinal absorption of the target protein molecule.

### Synthesis of Empty Salt Nano-Precipitates

The salt nano-precipitates of this invention can be prepared by firstly, providing a volume of the cation-providing inorganic metal salt solution. A volume of the anion-providing salt solution is separately provided.

The two volumes of salt solution are then mixed at a pre-determined volume ratio of parts of the cation-providing inorganic metal salt solution to 2 parts of the anion-providing salt solution. The solution is mixed until homogenous and incubated for 30 minutes at 37° C. for formation of the salt precipitate.

There is no harsh temperature or pH required during the precipitation of and/or subsequent protein loading into the salt nano-precipitates of this invention. This is a distinct advantage as protein molecules are prone to degradation or inactivation in harsh temperatures or pHs.

The synthesis conditions of the salt nano-precipitates of this invention can be easily modifiable depending on the protein molecule and target site of delivery. Modifying the synthesis conditions can also influence protein-loading capacity and the protein-release profile of the salt nano-precipitate. For example, modifying the salt particles with an organic molecule, such as citrate or alpha ketoglutarate, which carry carboxylic groups that can bind to the cations of the particles, can influence the binding and release of proteins.

Loading of Target Protein Molecule into Salt Nano-Precipitates

Protein loading may be performed by adding a volume of the target protein molecule to the cationic-providing inorganic metal salt solution. Subsequently, the anionic-providing salt solution is added to the mixture.

The solution is mixed until homogenous and incubated for 30 minutes at 37° C. for formation of the salt precipitate.

During in vitro testing, the inventors observed that the salt nano-precipitates have a high affinity with protein molecules.

For example, when assessing the affinity of insulin molecules towards barium sulphate (BaSO₄), barium sulphite (BaSO₃) and barium carbonate (BaCO₃) in vitro, the inventors observed that BaCO₃ showed the highest binding affinity for insulin (100% binding affinity) when compared to BaSO₄ and BaSO₃.

Further, the inventors observed that when the insulin-loaded BaSO₄, BaSO₃ and BaCO₃ were exposed to a wide range of pH in vitro, nearly 20%, 50% and 60% of insulin were released from BaSO₄, BaCO₃ and BaSO₃, respectively, when the respective insulin-salt precipitate complexes were exposed to pH of 2.47. At a pH of 1.78, only around 30% of insulin was released from the BaSO₄-insulin complexes and approximately 80% of insulin was released from the BaSO₃-insulin complexes.

This means that both BaSO₄ and BaSO₃ can protect insulin, to an extent, from degradation in stomach acidic pH, but that BaSO₄ is more effective than BaSO₃ in preventing insulin from degradation.

From FE-SEM analysis of insulin-loaded BaSO₃, BaCO₃ and BaSO₃, the inventors observed various changes in morphology of the insulin-loaded precipitates versus the empty precipitates i.e. insulin loading had clearly altered the shape of the nano-precipitates with different concentration of insulin resulting in different degree of structural changes in morphology.

At a low concentration of insulin (e.g. 2 μg), Insulin-loaded BaSO₄ particles were observed to be morphologically distinct from the empty particles in that the insulin-loaded particles are baseball shaped with a rough surface area and without a hollow structure. In contrast, as mentioned above, empty BaSO₄ particles have a round shape with a distinctive hollow structure in the middle and a smooth outer surface.

At increasing concentrations of insulin (e.g. 10 μg and 50 μg), the insulin-loaded BaSO₄ particles looked more elongated and the surface area appeared rougher. Notably, there were no empty SaSO₄ particles observed at high concentrations of insulin.

For the BaSO₃ particles, no noticeable changes of size, shape or surface morphology were observed between the insulin-loaded and the empty particles. However, the inventors observed very distinctive changes in aggregation pattern. As mentioned above, the empty BaSO₃ particles tended to clump together whereas the insulin-loaded particles were much less prone to aggregation, with their clusters clearly oval shaped.

Much like the BaSC₃ particles, there were also no noticeable changes of size, shape and surface morphology of insulin-loaded and empty BaCO₃ particles. Once again, the inventors observed very distinctive changes in aggregation patterns. While the empty BaCO₃ particles tended to clump together, the insulin-loaded BaCO₃ particles were much less prone to aggregation.

In an embodiment, the protein-loaded salt nano-precipitates can also be surface-modified with bio-adhesive proteins such as transferrin, casein and/or folate binding proteins. The inventors have demonstrated in the following Examples that doing so can help in increasing intestinal absorption of the protein-loaded precipitates, which in turn leads to increased absorption into the blood stream i.e. improved bioavailability.

### Therapeutic Use of Insulin-Loaded Sat Nano-Precipitates

The salt nano-precipitates of this invention are suitable for use in the oral delivery of protein therapeutics to animal or human subjects.

For example, insulin-loaded salt nano-precipitates such as barium sulphate (BaSO₄), barium sulphite (BaSO₃), barium carbonate (BaCO₃) and strontium sulphite (SrSO₃) are suitable for treating hyperglycemia, type 1 or type 2 diabetes melitus in an animal or a human subject.

During laboratory testing on an animal subject (streptozotocin (STZ)-induced diabetic male Wister Kyoto (WKY) rats), the inventors observed that all treatment groups of diabetic rats orally fed with insulin-loaded particles of BaSO₄, BaSO₃, BaCO₃ and SrSO₃, respectively, showed a reduction in blood glucose levels within 1 hour of oral administration. By comparison, diabetic WKY rats orally fed free insulin did not show any significant reduction of blood glucose levels at any point in time during a 4-hour long observation.

The inventors noticed during initial n v testing that a high salt concentration (20×) formulation of the salt nano-precipitates or this invention is distinctly more efficacious in reducing blood glucose levels in test subjects (diabetic rats) in comparison with a low salt concentration (1×) formulation, when orally administered. This could be because a 20× concentration formulation contained more adequate saturation of salt particles per volume, to more strongly bind insulin and to more efficiently act as a buffering agent in the acidic stomach environment.

As mentioned in a preceding section of the description, there is a distinct morphological difference between the low salt concentration (1×) and high salt concentration (20×) salt nano-precipitates. It is possible that the distinct morphology of the high concentration (20×) salt nano-precipitates also plays a part in the improved efficacy of the high concentration salt nano-precipitates in reducing blood glucose levels.

All in vivo tests explained below and in the Examples were conducted with the high salt concentration (20×) formulation.

For the group orally fed with insulin-loaded BaSO₄ particles, it was observed that the blood glucose level was significantly lowered compared to baseline levels within a time period of 1 to 3 hours (p<0.05). At the 4^{th} hour, the blood glucose level was still low relative to baseline level, but not significantly so and an ascending trend began to be observed.

For the group orally fed with insulin-loaded BaCO₃ particles, it was observed that there was a significant decrease of blood glucose levels within 1 to 2 hours. However, blood glucose levels started to show a slight increase at the 3^{rd} hour, and by the 4 hour, it was back to baseline levels.

For the group orally fed with insulin-loaded BaSO₃ particles, a reduction in blood glucose levels was observed but the reduction was not significant at the 4-hour mark.

For the group orally fed with insulin-loaded SrSO₃ particles, it was observed that there was a significant decrease in blood glucose levels in the beginning (even with a relatively low concentration of insulin 50 IU/kg), and this continued to be observed beyond the 4-hour mark.

Significantly, the inventors observed that the effect on blood glucose levels generated during oral administration of the above-mentioned insulin-loaded salt nano-precipitates of this invention was surprisingly similar to the effect generated by subcutaneous delivery of commercial human insulin aspart (Novorapid, Novonordisk). Insulin aspart, when administered subcutaneously, generally only lasts 4 to 5 hours, with a maximum drop in blood glucose levels during the 2 to 3-hour period.

As demonstrated in the following Examples, the inventors also surprisingly observed that the oral administration protocol that yields the best results with regards to reducing blood glucose levels involves pre-administration of bicarbonate orally before oral treatment with the insulin-loaded salt nano-precipitates of this invention. This is likely because the bicarbonate generated an elevated stomach pH level in the test subjects (diabetic rats), which resulted in an improved pH environment for passage of the salt nano-precipitates and subsequent absorption through the intestinal epithelium.

### Oral Delivery Forms of Protein-Loaded Salt Nano-Precipitates

The protein-loaded salt nano-precipitates of this invention may be processed into a suitable oral delivery dosage form, preferably, an oral liquid dosage form such as an oral suspension or an oral mixture, and, most preferably, an oral suspension.

The oral liquid dosage form may have a concentration of the protein-loaded salt nano-precipitates of this invention suspended in a suitable inert conventional carrier and/or diluent.

The protein-loaded salt nano-precipitates should be present in the oral liquid dosage form in a therapeutically suitable concentration of at least about 1 mg/ml and preferably, about 1 gm/ml.

A therapeutically suitable concentration is taken to mean a concentration of the protein-loaded nano-precipitates sufficient to achieve therapeutic effect when orally administered to an animal or a human subject

For example, when preparing an insulin-loaded salt nano-precipitate for oral delivery, a therapeutically suitable concentration means a concentration sufficient to achieve therapeutic effect when orally administered to an animal or a human subject suffering from hyperglycemia, type 1 or type 2 diabetes mellitus.

Hyperglycemia is defined as an excessively high blood glucose level, either in a fasting state (100 to 125 mg/dl) or in a non-fasting state (>180 mg/dl). Type 1 diabetes mellitus is defined as a form of diabetes in which very little or no insulin is produced by the pancreas resulting in high blood glucose levels. Type 2 diabetes mellitus is defined as a fasting blood glucose level exceeding 125 mg of glucose per dl of plasma.

As shown in the following Examples, the inventors have observed that a wide range of insulin concentrations can be loaded into the salt nano-precipitates of this invention, for example, from low concentration to high concentration e.g. from 1 to 100 IU/kg (IU/kg means 1 unit of insulin per kg of animal body weight).

### EXAMPLES

The following Examples illustrate the various aspects of a salt nano-precipitate of this invention. These Examples do not limit the invention, the scope of which is set out in the appended claims.

### Example 1: Synthesis of Empty BaSO₄, BaSO₃ and BaCO₃ Nano-Precipitates

This example illustrates the synthesis of empty BaSO₄ or BaSO₃ or BaCO₃ nano-precipitates.

Dulbecco's Modified Eagle's Medium (DMEM) powder was purchased from Invitrogen. Hydrochloric acid (HCl) (1M), sodium hydrogen carbonate (NaHCO₃), barium chloride dehydrate (BaCl₂·2H₂O), sodium sulphite (Na₂SO₃) and mucin were bought from Sigma Aldrich. Additionally, fluorescein isothiocyanate (FITC)-labelled insulin stock (human, recombinant, expressed in yeast, lyophilized powder) was purchased from Sigma Aldrich. HEPES (2-(4-(2-hydroxyethyl)-1-piperazinyl)ethanesulfonic acid) and sodium carbonate (Na₂CO₃) were from Fisher Scientific and sodium sulphate (Na₂SO₄) from Merck. Insulin aspart (NovoRapid®), Novo Nordisk) was purchased from a local pharmacy. Pepsin was purchased from Promega (USA).

1M of BaCl₂, Na₂SO₄, Na₂SO₃ and Na₂CO₃ stock solutions were prepared by calculating amount of the respective molecular weight of powder and dissolving them in water. All solutions were stored in 1 mL aliquots at −20° C. 100 mL of bicarbonated DMEM solution was freshly prepared by dissolving 1.35 g of DMEM powder in 95 mL pure Milli-Q water, followed by the addition of 0.37 g of sodium hydrogen carbonate (0.44 mM final concentration). The pH of the solution was then adjusted to the desired level by addition of either 1M Ha or 1M NaOH. Final volume was then adjusted to 100 mL, 2 mg/mL of FITC-insulin stock solution was prepared by dissolving the FITC-insulin powder into an appropriate volume of pure Milli-Q® water.

Each Ba salt precipitate (BaSO₄, BaSO₃ or BaCO₃) was prepared by incorporating a volume of cation-providing BaCl₂ salt into either a volume of HEPES-buffered solution (pH adjusted to 8.0) or milliQH2O and mixing the resultant solution with a volume of one of an anion-providing salt i.e. Na₂SO₄, Na₂SO₃ or Na₂CO₃, respectively. The final mixture was incubated for 30 minutes at 37° C. and subsequently added to miliQH2O to obtain the final volume of particle suspension.

Table 1 below shows the various concentrations of reacting salts used to synthesize the three different Ba salt precipitates.

TABLE 1
Various concentrations of reacting salts
for synthesis of BaSO₄, BaSO₃ or BaCO₃

| | Ba salt precipitates |
|---|---|
| | 1M BaCl₂ (5 μl) |
| 1M Na₂SO₄ (2 μl) | BaSO₄ |
| 1M Na₂SO₃ (2 μl) | BaSO₃ |
| 1M Na₂CO₃ (2 μl) | BaCO₃ |

20X concentrated Ba precipitate formulations prepared for in vivo use

| | 1M BaCl₂ (100 μl) |
|---|---|
| 1M Na₂SO₄ (40 μl) | BaSO₄ |
| 1M Na₂SO₃ (40 μl) | BaSO₃ |
| 1M Na₂CO₃ (40 μl) | BaCO₃ |

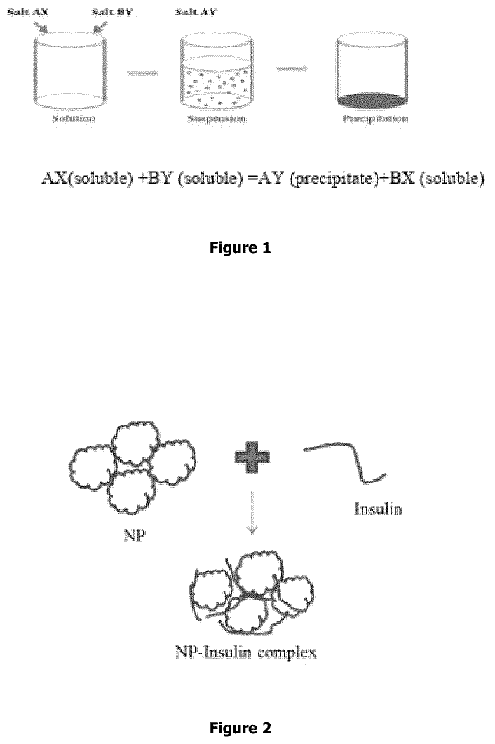

FIG. 1 shows a simple diagrammatic representation of the precipitation reaction.

### Example 2: Insulin Loading into BaSO₄, BaSO₃ and BaCO₃ Nano-Precipitates

This example illustrates protein (insulin) loading of BaSO₄ or BaSO₃ and BaCO₃ nano-precipitates.

Three Ba salt precipitates (BaSO₄, BaSO₃ or BaCO₂) were prepared as per Example 1 i.e. by incorporating a volume of cation-providing BaCl₂ salt into either a volume of HEPES-buffered solution (pH adjusted to 8.0) or miliQH2O and mixing the solution with a volume of one of an anion-providing salt i.e. Na₂SO₄, Na₂SO₃ and Na₂CO₃, respectively. Insulin was introduced right before addition of the second salt to the medium. The final mixture was incubated for 30 minutes at 37° C. and subsequently added to millQH2O to obtain the final volume of particle suspension.

Table 2 below shows the various concentrations of reacting salts and insulin used for preparation of the insulin-loaded Ba salt precipitates.

TABLE 2
Various concentrations of reacting salts and insulin
to prepare insulin-loaded BaSO₄, BaSO₃ or BaCO₃

| | Insulin | 1M BaCl₂ (5 μl) |
|---|---|---|
| 1M Na₂SO₄ (2 μl) | FITC-insulin (2 mg/ml, 1-25 μl)/ | BaSO₄ |
| | Insulin Aspart (1-5 IU/kg) | |
| 1M Na₂SO₃ (2 μl) | FITC-Insulin (2 mg/ml, 1-25 μl)/ | BaSO₃ |
| | Insulin Aspart (1-5 IU/kg) | |
| 1M Na₂CO₃ (2 μl) | FITC-insulin (2 mg/ml, 1-25 μl)/ | BaCO₃ |
| | Insulin Aspart (1-5 IU/kg) | |

20X concentrated Ba precipitate formulations loaded with
Insulin Aspart for in vivo use

| | Insulin | 1M BaCl₂ (100 μl) |
|---|---|---|
| 1M Na₂SO₄ (40 μl) | Insulin Aspart (100 IU/kg) | BaSO₄ |
| 1M Na₂SO₃ (40 μl) | Insulin Aspart (100 IU/kg) | BaSO₃ |
| 1M Na₂CO₃ (40 μl) | Insulin Aspart (100 IU/kg) | BaCO₃ |

The 20× concentration (high salt concentration) Ba salt precipitates prepared as per Table 2 above were used in all the following in vivo testing Examples.

FIG. 2 shows a simplified diagrammatic depiction of insulin loading of the Ba salt precipitates.

### Example 3: Characterization and Morphological Screening of BaSO₄ BaSO₃ and BaCO₃ Nano-Precipitates

### Fourier Transform Infrared Spectroscopy (FT-IR)

A volume of 250 μl of 1M of BaCl₂ was taken in a centrifuge tube, followed by addition of 100 μl of 1M Na₂SO₄ or Na₂SO₃ or Na₂CO₃ to the respective tubes to prepare BaSO₄. BaSO₃ and BaCO₃ precipitates. The samples were then incubated at 37° C. for 30 minutes. The final volume of the solution was made to 50 ml with addition of miliiQ2O. Samples were then centrifuged for 15 minutes at RPM 5,000. Precipitates were separated from the supernatant, stored sequentially at −20° C. overnight and at −80° C. for 0.5 hour and finally placed into a freeze-dryer (Labconco freeze dryer, Kansas City, MO, USA) for 5 hours. Samples were read using a Varian FT-IR (Santa Clara, CA, USA) and analysed with Resolution Pro 640 software.

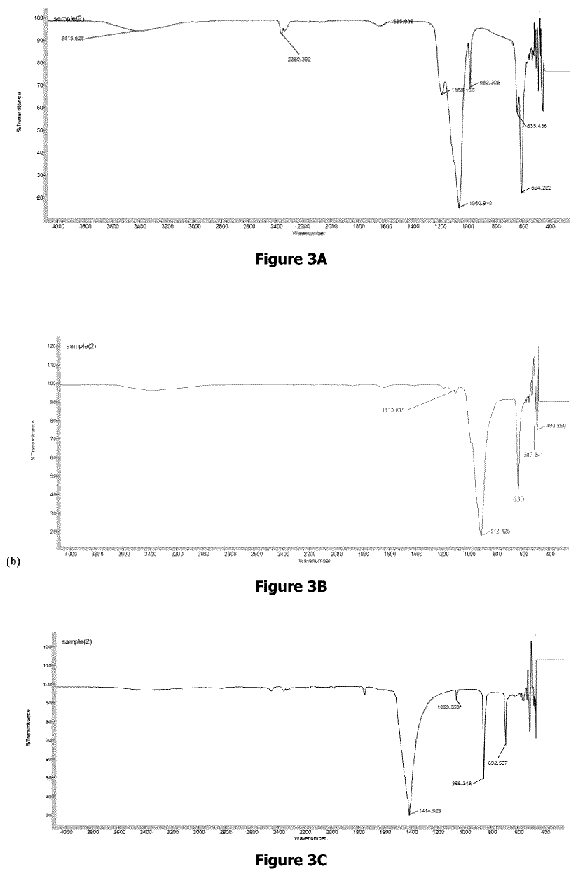

FT-IR was used to confirm formation of the Ba salt precipitates BaSO₄, BaSO₃ and BaCO₃) by revealing the functional groups. FIG. 3 shows the FT-R spectrum obtained for the three different Ba salt precipitates.

The IR spectrum for BaSO₃ (FIG. 3A) shows peaks at 118 cm⁻¹, 1060 cm⁻¹ and 982 cm⁻¹, which are attributed to symmetric stretching of SO₄²⁻, peaks at 1635 cm⁻¹ due to stretching vibration of SO₄²⁻ and peaks at 604 cm⁻¹ and 637 cm⁻¹, representing out-of-plane bending for SO₄²⁻. Peaks at 2360 cm¹ seem to be the overtone of S—O vibration (SFontes A. 8. et. al. (2015)¹⁷, Ramaswamy V. et. al, (2010)¹⁸). Existence of the functional group suggests the generation of BaSO₄ particles.

FIG. 38 shows the characteristic peaks for SO₃²⁻ found at 490 cm⁻¹, 630 cm⁻¹, 912 cm⁻¹ and 1133 cm⁻¹ position, suggesting the formation of BaSO₃ particles (Qiao X. et. al. (2018)¹⁹).

The characteristic peaks for CO₃²⁻ found at 855 cm⁻¹ and 692 cm⁻¹ (FIG. 3C) were owing to in-plane and out-of-plane bending, whereas the peak at 1414 cm⁻¹ corresponds to the asymmetric stretching of C—O bond and peak at 1059 cm⁻¹ is attributed to the symmetric C—O stretching vibration (Sreedhar 8, et. al. (2012)²⁰), thus indicating the formation of BaCO₃ particles.

Elemental analysis of particles by Energy Dispersive X-Ray Spectroscopy (EDX)

5 μl of cation-providing salt was added to 20 μl milliQH2O, followed by addition of 2 μl of anion-providing salt. After 30 minutes of incubation at 37° C., 1 ml of milliQ H₂O was added, 2 μl of the solution containing the particles was transferred on a coverslip. The coverslip was then air-dried in elevated temperature (45° C.) inside a dryer for 1 hour. The dried sample was marked with a circular line with a marker for easy identification under a microscope. Samples were then subjected to platinum sputtering for 45 seconds with 30 mA and factor of 2.3. Visualizations and documentation were done using a Field Emission Scanning Electron Microscope (FE-SEM) (Hitachi/SU8010, and Tokyo, Japan) at 5.0 kv.

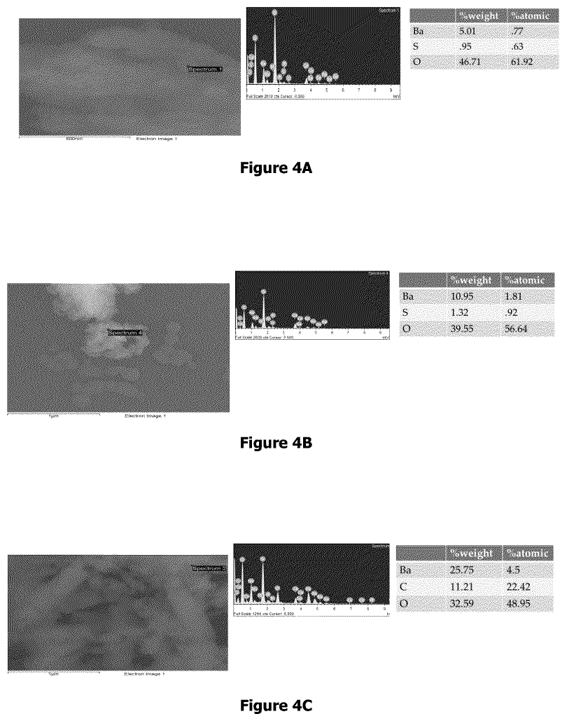

EDX was carried out for elemental analysis of Ba salt particles (FIG. 4). The presence of the desired elements in the samples confirm formation of the respective particles. Elemental analysis of BaSO₄ (FIG. 4A) and BaSO₃ (FIG. 4B) shows the presence of Ba, S and O, whereas analysis of BaCO₃ confirms the presence of Ba, C and O (FIG. 4C).

### Example 4: Particle Stability Assessment of BaSO₄, BaSO₃ and BaCO₃ Nano-Precipitate

One of the biggest challenges in oral delivery is pre-systemic degradation due to harsh pH in the stomach environment, which has a fluctuating pH and can be extremely acidic (˜pH 1) when the subject is in a lasting state. Any oral delivery agent needs to be resistant to extreme acidic pH to survive gastrointestinal transport. A set of in vivo experiments were designed and carried out to test the stability of the BaSO₄, BaSO₃ and BaCO₃ particles in different pHs, by exposing the formulations to a wide range of pHs for a period of 3 hours.

BaSO₄, BaSO₃ and BaCO₃ nano-precipitates were prepared as per Example 1. After 30 minutes of incubation at 37° C., 1 ml of DMEM of different pHs (pH 7.78 to pH 1.78) was added to each sample and the samples were then kept at 37° C. The samples were prepared in triplicates and measured at 320 nm at different time points over a period of 3 hours.

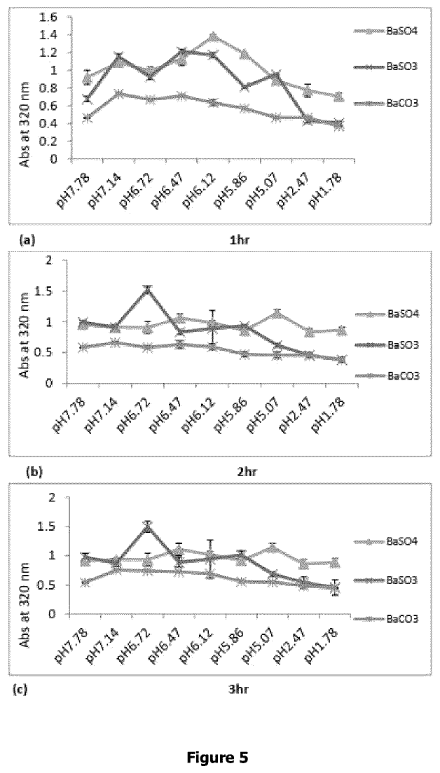

FIG. 5 shows changes in the turbidity pattern at 320 nm for all three Ba salt particles. Both BaSO₄ and BaSO₃ particles demonstrated much higher absorbance than BaCO₃ particles when measured immediately after their generation at pH 7.78, reflecting the tendency of BaSO₄ and BaSO₃ to generate significantly higher number of particles than BaCO₃.

Consistently high turbidity values for BaSO₄ particles indicate their excellent resistance to degradation over a wide range of pHs at different time points (1 to 3 hours), white the turbidity of BaSO₃ particles sharply dropped at pH 1.69 suggesting a considerable amount of particle loss. BaCO₃ maintained almost the same level of turbidity throughout the range of pHs, with apparently no significant particle loss at low pH.

### Example 5: Insulin Loading Efficiency of BaSO₄, BaSO₃ and BaCO₃ Nano-Precipitates

This example illustrates the protein (insulin) loading efficiency of BaSO₄ or BaSO₃ and BaCO₃ nano-precipitates.

FITC-insulin-loaded BaSO₄, BaSO₃ and BaCO₃ were prepared as per Example 2. After 30 minutes of incubation at 37° C., 200 μl of DMEM prepared at different pHs (pH 7.78 to pH 1.78) was added. After incubation at room temperature for 10 minutes, the samples were centrifuged at 6000 rpm for 2 minutes at 4° C. The supernatant was discarded and the resultant pellet was washed with 50 μl of HEPES, 2×. At the end of washing, the supernatant was again discarded and the pellet carrying insulin-bound particles was re-suspended with 200 μl of 10 mM EDTA. 200 μL of supernatant was carefully transferred into a black 96-well plate (PerlinElmer Opti-Plate™-96 F) and fluorescence intensity values were recorded using a fluorescence micro-plate reader (PerKinElmer Victor X5 2030 Multi-label Reader) set with excitation/emission filters at 485 nm/535 nm wavelengths. A standard curve was prepared from absorbance values for known amounts of free PITC-Insulin (0, 400, 800, 1200, 1600, 2000, 4000 ηg) added to DMEM. % binding at pH 7.78 was calculated from the formula given below.

$$\% binding = \frac{F I T C - Insulin conc . in pallet}{conc . of added F I T C - Insulin} \times 100$$

Percentage of release at subsequent pHs (7.18 to 1.78) was calculated by subtracting the amount of FITC-insulin found in the pellet from the amount of FITC-insulin added initially and finally, multiplying by 100.

The affinity of insulin molecules towards BaSO₄, BaSO₃ and BaCO₃ was assessed by separating FITC-conjugated insulin-loaded particles from free fluorescent insulin by centrifugation. Fluorescence intensity was measured at 485 nm/535 nm (excitation/emission) wavelengths. The amount of FITC-insulin present in the pellet was calculated from the standard curve prepared with known amount of FITC-insulin versus respective absorbance. The percentage of loading was calculated by dividing the amount of FITC-insulin present in pellet with the amount initially added and then multiplying with 100.

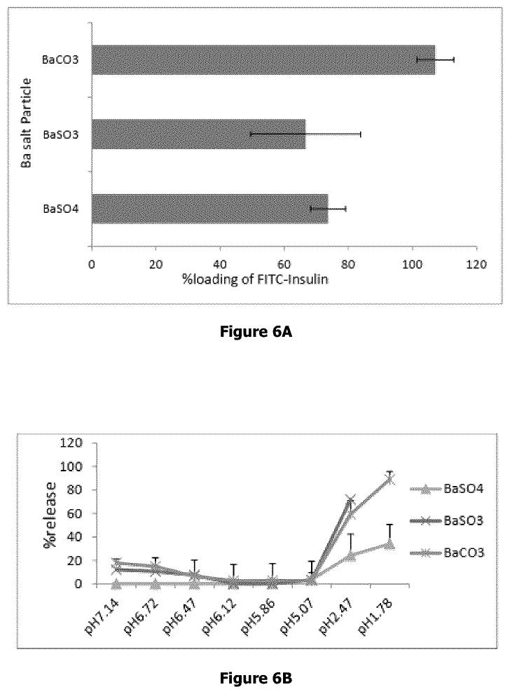

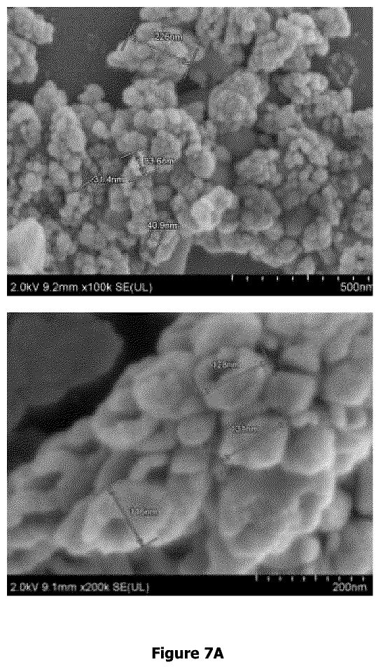

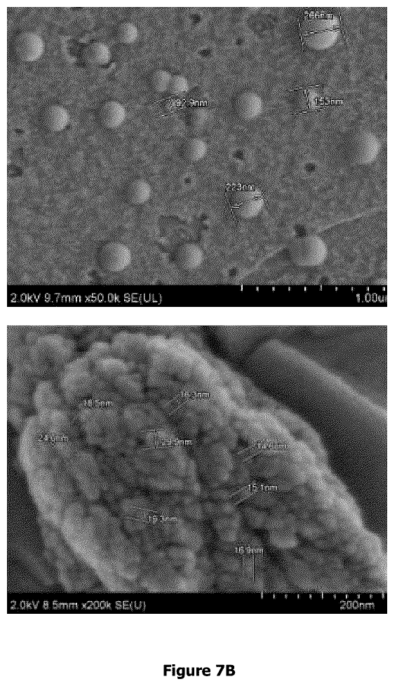

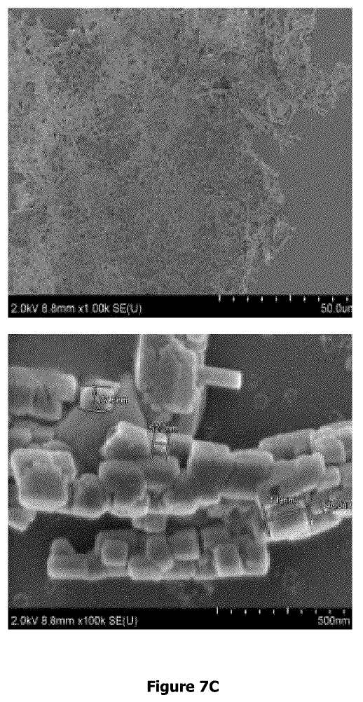

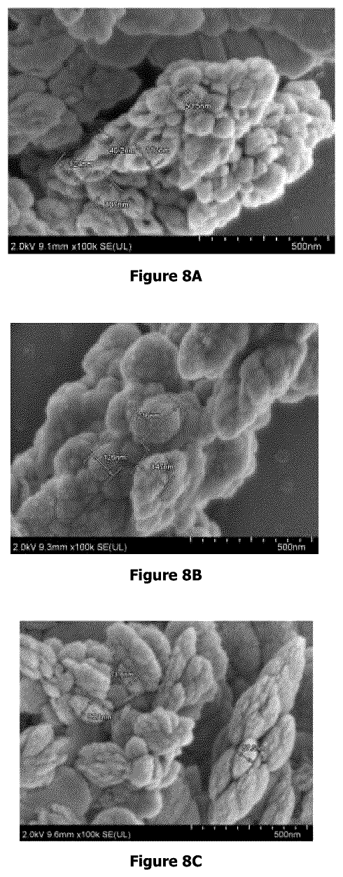

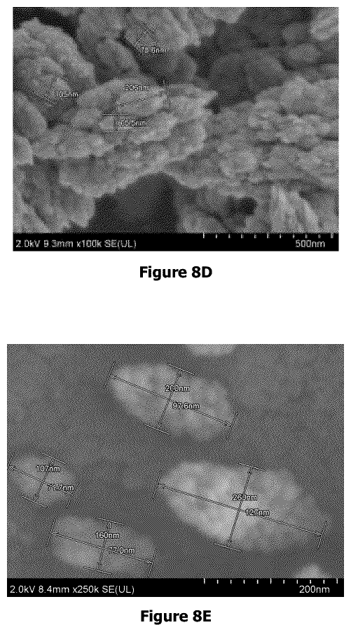

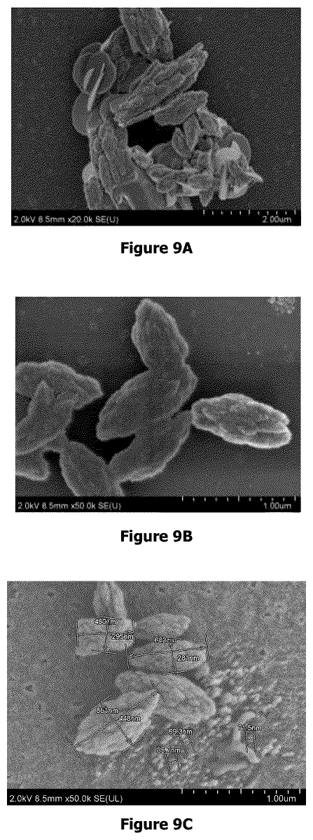

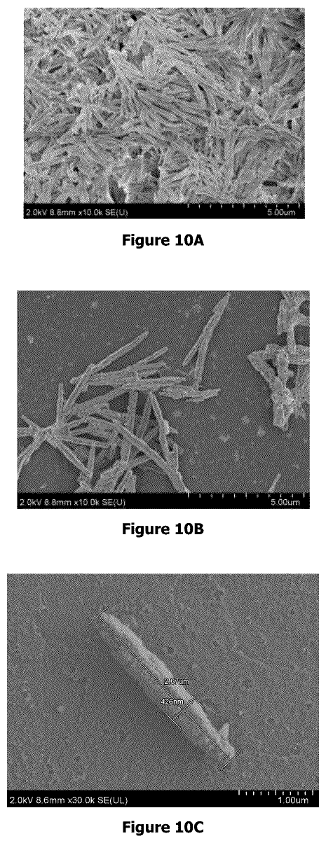

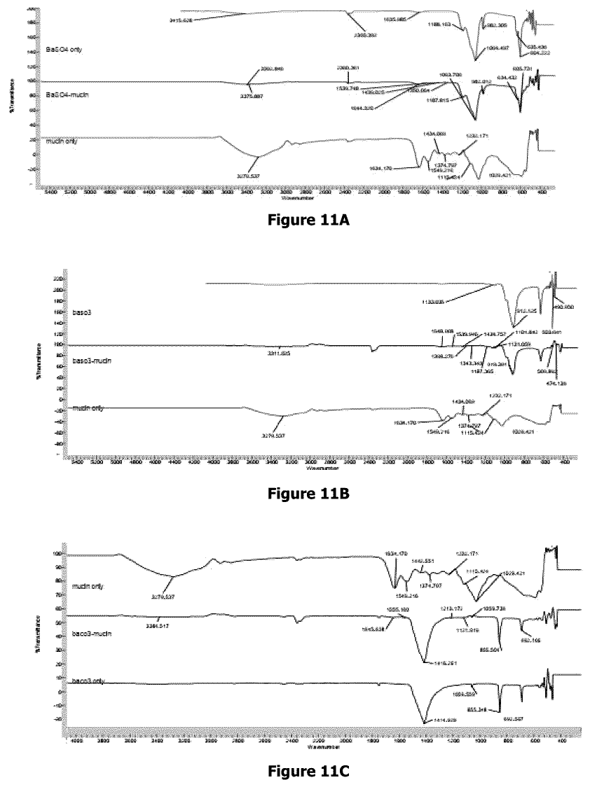

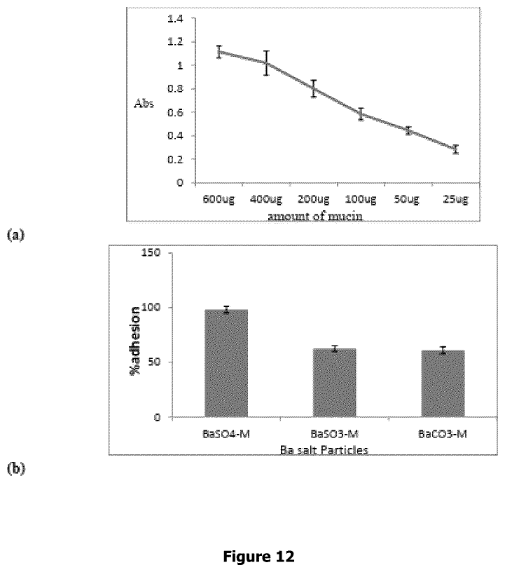

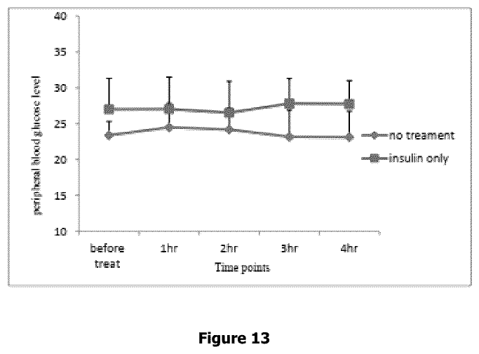

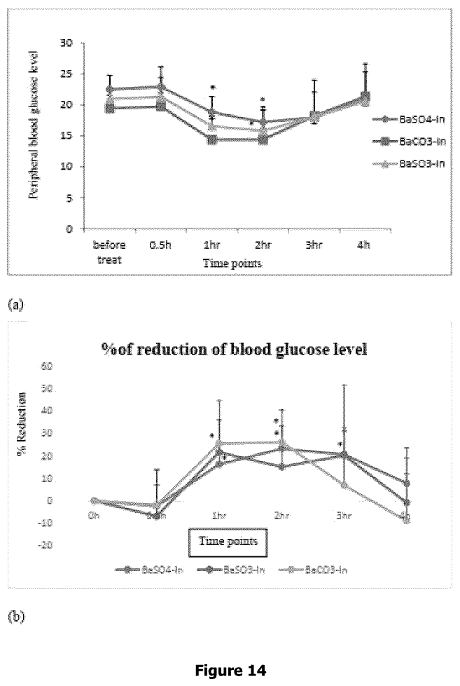

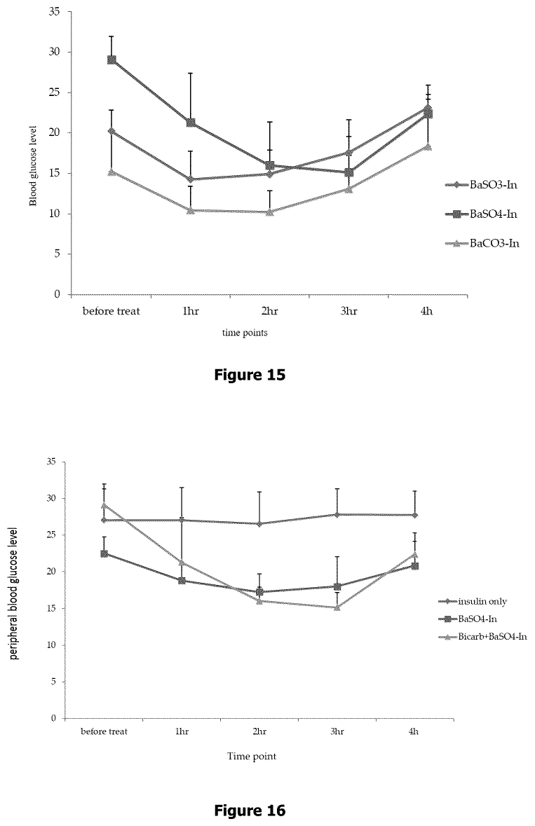

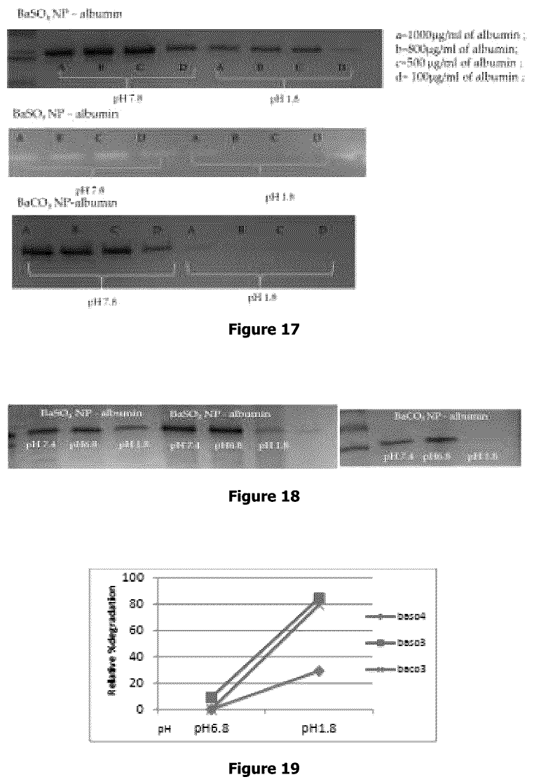

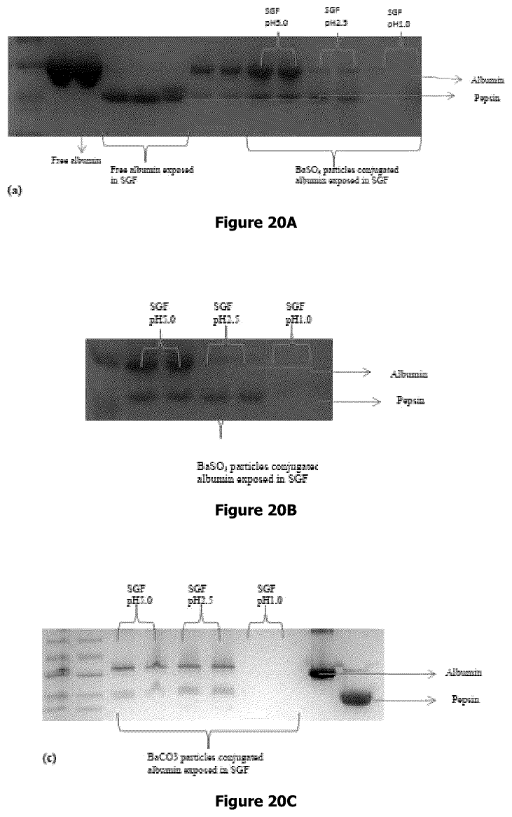

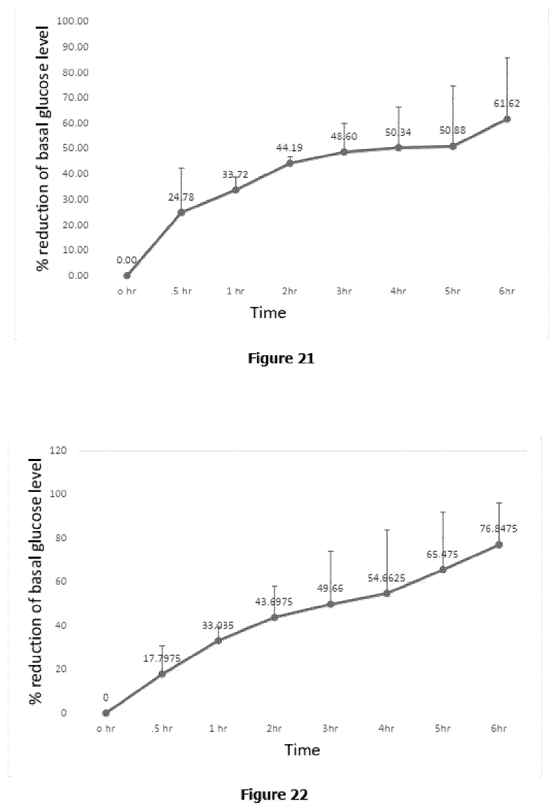

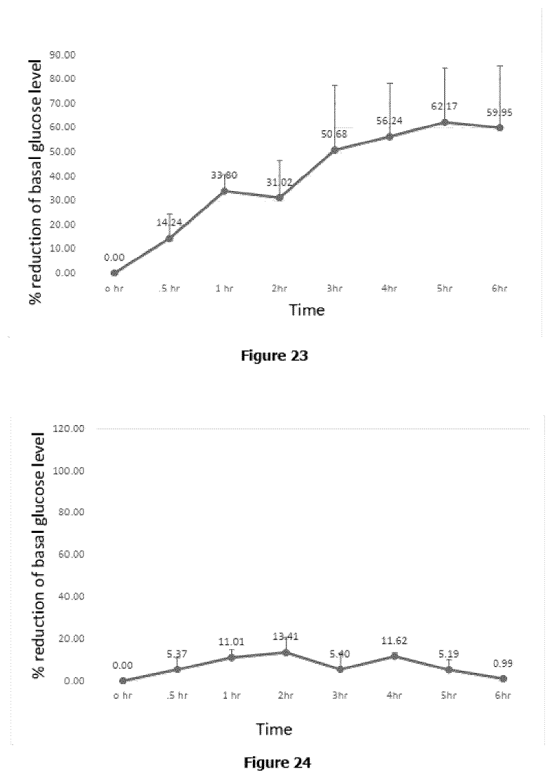

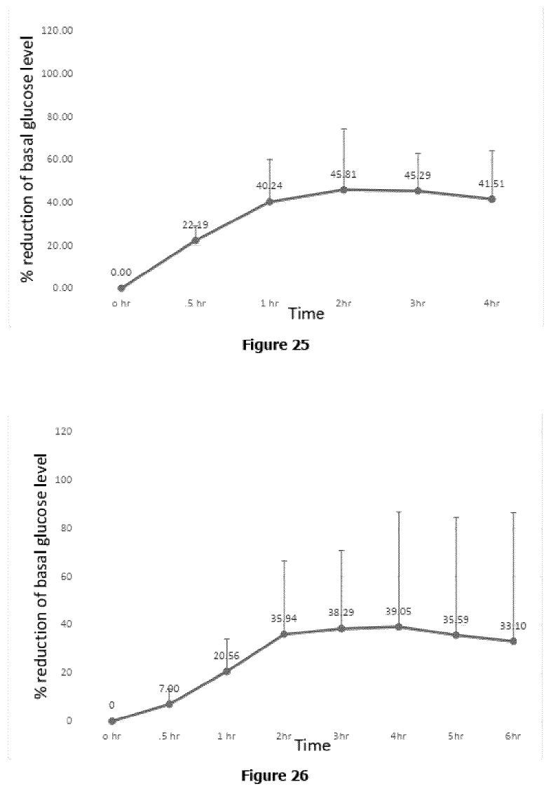

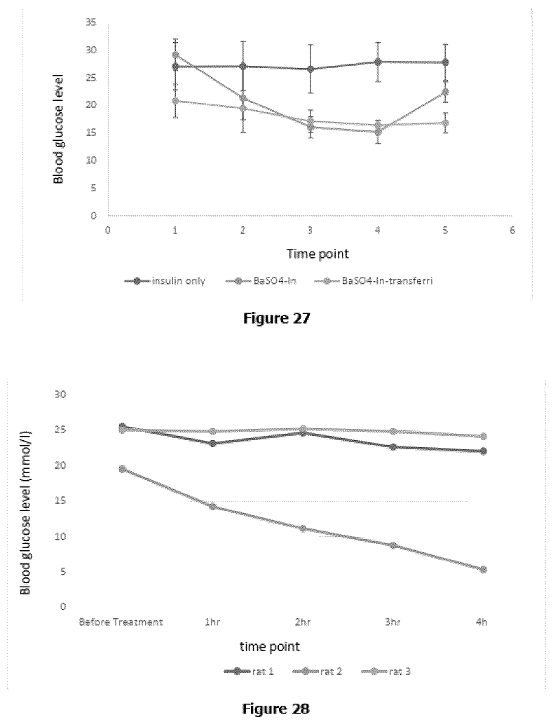

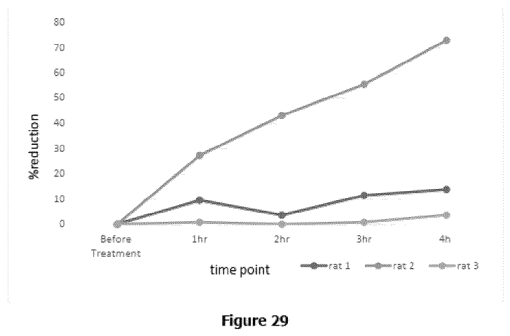

FIG. 6A shows the percentage of insulin binding to BaSO₄, BaSO₃ and BaCO₃. BaCO₃ particles demonstrated a higher binding affinity for insulin (100%) than BaSO₄ and BaSO₃ particles. The equations used to calculate the percentage of loading and percentage of release of insulin are shown below.

$$\% loading = \frac{F I T C - Insulin conc . in pellet}{conc . of added F I T C - Insulin} \times 100$$

$$\% release = \frac{\begin{matrix}( F I T C - insulin conc . in pellet synthesized at pH 7.78 - \\ F I T C - insulin conc . in pellet of particles synthesized \\ at subsequent pH ) \times 100\end{matrix}}{FITC - insulin conc . in pellet of N P synthesized at pH 7.78}$$

FITC-insulin-loaded particles were exposed to a wide range of pHs, and as shown in FIG. 6B, nearly 20%, 50% and 60% of insulin were released from BaSO₄, BaCO₃ and BaSO₃ particles, respectively, when the respective insulin-particle complexes were exposed to pH of 2.47. At a pH of 1.78, only around 30% of insulin was released from BaSO₄ and approximately 80% of insulin was released from BaSO₃ particles.

### Morphological Assessment of Empty and Insulin-Loaded Particles by FE-SEM

BaSO₄, BaSO₃ and BaCO₃ were similarly prepared as described in Example 3 for FE-SEM and EDX analysis. Insulin-loaded salt particles were prepared separately (as per Example 2). Visualization and documentation were done using FE-SEM (Hitachi/SU8010, Tokyo, Japan) at 2.0 kv.

FE-SEM analysis was carned out to study the particle morphology with respect to particle size, shape and aggregation pattern with and without insulin loading into the salt particles.

FIG. 7 shows micrographs for BaSO₄, BaSO₃ and BaCO₃. The BaSO₄ particles (FIG. 7A) possessed distinctive irregular, oval to hollow rounder shape, with the bigger particles having oval to hollow rounder shape with particle size between 110 to 200 nm and the smaller ones being irregular shaped with particle size between 30 to 100 nm. The BaSO₃ particles (FIG. 7B) also had the prominent distribution of big (90 to 270 nm) and very small (15 to 40 nm) sized particles. The particles were found to be rounder shaped and held in tight baseball shaped clusters with rough surface area. Each distinctive particle duster ranged from 500 nm to 1.0 μm in diameter. Larger particles displayed a rounder shape with smooth surface area, without forming a duster. The BaCO₃ particles (FIG. 7C) demonstrated a distinctive square to rectangular shaped morphology, with the particle size varying widely from 50 to 500 nm. The particles were closely aggregated with one another, forming rod or filament like structures.

FIGS. 8 to 10 show the scanning electron microscopy images of empty BaSO₄, BaSO₃ and BaCO₃ particles and insulin-loaded BaSO₄, BaSO₃ and BaCO₃ particles (IX low salt concentration and 20× high salt concentration, as per Table 2).

Effect of Insulin Loading into BaSO₄ Particles

Insulin was loaded into the BaSO₄ particles in varying amounts. Loading of insulin clearly altered shape of the particles, with different concentrations of insulin showing different degrees of structural changes in particle morphology (FIG. 8).

FIG. 8A shows that free BaSO₄ particles are of a rounder shape with a distinctive hollow structure in the middle and an apparently smooth outer surface. FIG. 8B shows BaSO₃ particles prepared with 2 μg of initially added insulin, giving the impression of free particles residing with insulin-loaded particles which were morphologically different from the empty particles. Insulin-loaded particles are baseball shaped with a rough surface area and without a hollow structure. FIGS. 8C and 8D show insulin-loaded BaSO₃ particles prepared with 10 μg and 50 μg of insulin. With increasing insulin concentrations, the particles looked more elongated and the surface area appeared rougher. Interestingly enough, there was no empty BaSO₄ particle visualized at high concentrations of insulin.

FIG. 8E shows high salt concentration (20×) insulin-loaded BaSO₄ particles. As can be seen, the high salt concentration particles are morphologically distinct from low salt concentration particles (FIGS. 8B, 8C and 8D) with only smaller round shape particles observed.

Effect of Insulin Loading into BaSO₃ Particles

FIG. 9 shows the morphological features of empty and insulin (FITC-insulin)-loaded BaSO₃ particles. There were no noticeable changes in terms of size, shape or surface morphology. However, very distinctive changes in aggregation pattern were observed. Empty particles tended to clump together whereas insulin-loaded particles were much less aggregated, with their clusters clearly oval shaped.

From FIG. 9C, it can be seen that high salt concentration (20×) insulin-loaded BaSO₃ particles are morphologically distinct from the low salt concentration particles (FIG. 9B) with relatively less aggregation observed and formation of clusters that look irregular.

Effect of Insulin Loading into BaCO₃ Particles

FIG. 10 shows morphological features of empty and insulin-loaded BaCC particles with no noticeable changes in size, shape and surface morphology of individual particles. However, very distinctive changes in aggregation pattern were observed. Free particles tended to clump together whereas insulin-loaded particles looked much less aggregated.

FIG. 10C slows that high salt concentration (20×) insulin-loaded BaCO₃ particles are morphologically distinct from the low salt concentration particles (FIG. 10B) with long, thin threads that appear to be inter-tangled with each other, observed.

### Example 6: Assessment of Adhesion of BaSO₄ BaSO₃ and BaCO₃ Nano-Precipitates to Mucin

This example illustrates the adhesion of BaSO₄ or BaSO₃ and BaCO₃ nano-precipitates to mucin.

### Spectrophotometric Analysis of Particle Adhesion to Mucin

The BaSO₄, BaSO₃ and BaCO₃ particles were prepared as per Example 1. 200 μl of milliQH₂O was added followed by addition of 100 μl of mucin (3 g/L). The mixture was then incubated at 37° C. for 10 minutes followed by centrifugation at RPM 13, for 5 minutes. The supernatant was collected in a fresh tube. 250 μl of Bradford agent was directly added to the tube. The mixture was then incubated for 5 minutes before transferring to 96 well microplates.

Absorbance was read at 595 nm using victor X5 spectrophotometer (PerkinElmer, USA). A standard curve was created using a series of different concentrations of mucin solution of 25 to 600 μg, mixed with 250 μl of Bradford agent. Unknown amount of mucin present in the supernatant was calculated from the standard curve. The percentage of adhesion of Ba salt particles to mucin was calculated from added mucin with the two-step formula shown below.

$$Mucin bound to Particles = mucin present in the pallet = added mucin - mucin from supernatant$$

$$\% adhesion = \frac{mucin bound to particles \times 100}{added mucin}$$

FT-R Assessment of BSO₄, BaSO₃ and BaCO₃ Adhesion to Mucin

In vivo tests were done to determine the affinity of BaSO₄, BaSO₃ and BaCO₃ particles to mucin in order to predict whether the particles would be capable of crossing the intestinal lining. The data from FT-IR analysis would indicate any possible interactions of mucin molecules with the particles.

BaSO₄, BaSO₃ and BaCO₃ samples were prepared in bigger volumes for this experiment. 250 μl of 1M of BaCl₂ in a 50 ml tube was mixed with 100 μl of 1M Na₂SO₄ or Na₂SO₃ or Na₂CO₃ to prepare BaSO₄ or BaSO₃ or BaCO₃ particles. The samples were then incubated at 37° C. for 30 minutes. Sample tubes were then topped up to 25 ml with addition of milliQH₂O, followed by addition of a solution containing 5 ml of mucin (3 g/l). Samples were centrifuged for 15 minutes at 5,000 RPM. Precipitates were then stored at −20° C. overnight, followed by storing at −80° C. for 0.5 hour and freeze-drying (Labconco freeze dryer, Kansas City, MO, USA) for 5 hours. Varian FT-tR (Santa Clara, CA, USA) and Varian Resolution Pro 640 software (Santa Clara, CA, USA) were used to check the spectra of particles alone, particle-mucin complexes and mucin alone.

FT-IR was used to confirm mucin binding of BaSO₄, BaSO₃ and BaCO₃ particles by revealing the functional groups for each particle along with the mucin protein. Samples for particles only, mucin only and particles adhered to mucin were analyzed with FT-IR. The comparison of the peak patterns showed positive mucin binding for BaSO₄, BaSO₃ and BaCO₃ particles. Free mucin displayed characteristic peaks of N—H at 3279 cm⁻¹, Amide I at 1634 cm⁻¹, Amide 11 at 1549 cm⁻¹, C—H at 1434 cm⁻¹ and 1374 cm⁻¹, Amide II 1232 cm⁻¹, C—O—C at 1115 cm⁻¹ and C—C—O at 1029 cm⁻¹ (Liu F. et, al. (1988)²²).

FIG. 11A shows the FT-IR spectrum of BaSO₄ particles alone, free mucin and BaSO₄ particle-mucin complexes. BaSO₄ particles showed 8 characteristic peaks, as described above in Example 3. Mucin displayed 8 characteristic peaks (as stated above). Mucin-adhered BaSO₄ particles demonstrated 12 distinctive peaks, with peaks 2360 cm⁻¹, 118 cm⁻¹, 1064 cm⁻¹, 982 cm⁻¹, 635 cm⁻¹ and 605 cm⁻¹ matching the peaks of BaSO₄ particles in the similar positions. The peak at 3415 cm⁻¹ showed a deviation to 2392 cm⁻¹ probably due to mucin attachment to the particles. Similarly, mucin peaks at 3279 cm⁻¹, 1549 cm⁻¹ and 1434 cm⁻¹ seemed to be shifted to 3275 cm⁻¹, 1539 cm⁻¹ and 1439 cm respectively in mucin-attached particles. Existence of characteristic peaks for BaSO₄ and muon indicates interactions between BaSO₄ particles to mucin.

FIG. 11B shows the FT-IR spectrum for free BaSO₄ particles, free mucin and BaSO₄-mucin complex. BaSO₄ particles possess 4 characteristic peaks as described in Example 3. Mucin has 8 characteristic peaks (as mentioned above). Mucin-adhered BaSO₃ particles showed 13 distinctive peaks. The peaks of BaSO₃ particles, 1133 cm⁻¹, 912 cm⁻¹, 630 cm⁻¹ and 409 cm⁻¹ were found at 1131 cm⁻¹, 919 cm⁻¹, 509 cm⁻¹ and 474 cm⁻¹ positions in mucin-adhered BaSO₃ particles. 8 characteristic peaks from mucin were found at 3311 cm⁻¹, 1648 cm⁻¹, 1539 cm⁻¹, 1434 cm⁻¹, 1298 cm⁻¹, 1343 cm⁻¹, 1187 cm⁻¹ and 1101 cm⁻¹ positions. Mucin peaks showed deviation from their positions, likely due to its binding to BaSO₃ particles. The presence of characteristic peaks for BaSO₃ particle as well as for mucin indicates positive adhesion of BaSO₃ particles to mucin.

FIG. 11C shows the FT-IR spectrum for free BaCO₃ particles, free mucin and BaCO₃-mucin complex. BaCO₃ particles showed 4 characteristic peaks as described in Example 3. Mucin-adhered BaCO₃ particles demonstrated 9 distinctive peaks. Mucin characteristic peaks at 3279 cm⁻¹, 1634 cm⁻¹, 1549 cm⁻¹, 1232 cm⁻¹, 1115 cm₋₁ were found respectively at 3384 cm⁻¹, 1645 cm⁻¹, 1555 cm⁻¹, 1213 cm⁻¹, 1121 cm⁻¹ positions in mucin-adhered BaCO₃ particles. The presence of characteristic peaks for BCO₃ particles and mucin indicates positive adhesion of BaCO₃ particles to mucin.

Mucin adhesion to the particles was further assessed by spectrophotometric protein quantification. The percentage of mucin adhesion to BaSO₄, BaSO₃ and BaCO₃ particles was calculated by measuring the protein content (mucin) precipitated out with the particles after centrifugation. Protein quantification was done by the Bradford method. The standard curve was prepared with a series of known amount of mucin versus respective absorbance values.

FIG. 12 shows the Bradford assay data for mucin adhesion, where (a) shows the standard curve prepared from acetate buffer solution (ABS) versus a known amount of mucin, whereas (b) reveals the percentage of adhesion to BaSO₄, BaSO₃ and BaCO₃ particles. The BaSO₃ particles showed the highest mucin adhesion (100%), followed by BaSO₃ and BaCO₃ pantiles with 60 to 70% of mucin adhesion.

### Example 7: Oral Administration of Insulin-Loaded BaSO₄, BaSO₃ and BaCO₃ Particles to Diabetic Animals and Management of Hyperglycemia

This example illustrates the effect of orally administered protein (insulin) loaded BaSO₄ or BaSO₃ and BaCO₃ nano-precipitates on hyperglycemia.

Induction of Diabetes in Rats with Streptozotocin (STZ)

8 to 12 weeks-old male healthy Wister Kyoto rats (WKY) were subjected to intraperitoneal (IP) injection of STZ (65 mg/kg). A fraction of the rats developed hyperglycemia with persistent high level of peripheral blood glucose (>13 mM) within a week or two. Glucose levels in the blood (collected via tail vein) were measured using a glucometer (Terumo, Japan). The rats that did not develop diabetes with the first injection were given a second IP dose of 65 mg/kg STZ.

Effect of Orally Administrated Insulin-Loaded BaSO₄, BaSO₃ and BaCO₃ Particles on Hyperglycaemia

Short acting human insulin analogue, Insulin Aspart (NovoRapid, Novonordisk)-loaded BaSO₄, BaSO₃ and BaCO₃ particles were administrated to ST2-induced diabetic male Wister Kyoto (WKY) rats. For oral administration, particles were prepared with a high concentration of reactant salts (20× higher than that used in original formulations Table 2) were loaded with a high dose of insulin Aspart (100 IU/kg).

Rats showing a clear sign of diabetes (peripheral blood glucose level of ≥13 mM) were divided into different control and treatment groups. Each group consists of 3 animals. After taking a baseline blood glucose reading, each rat was given insulin Aspart-loaded BaSO₄, BaSO₃ and BaCO₃ formulations via oral gavage. Rats from the control group were kept untreated (negative control), whereas rats from the positive control group received a solution containing free insulin (100 IU/kg).

Treatment groups received 500 μl of solution containing insulin-loaded BaSO₄, BaSO₃ and BaCO₃ particles by oral gavage. Blood glucose was read at regular time intervals (0.5 hour, 1 hour, 2 hour, 3 hour, and 4 hour). Blood glucose level at “0” hour is the baseline blood glucose reading taken right before oral delivery of insulin-loaded BaSO₄, BaSO₃ and BaCO₃ particles. The percentage of reduction at any given point of time was calculated from the baseline blood glucose level using the formula below.

$$\% reduction of blood glucose level = \frac{\begin{matrix}( baseline blood glucose read - blood glucose \\ reading at any point of time ) \times 100\end{matrix}}{baseline blood glucose level}$$

FIG. 13 shows the data from control groups, i.e. no treatment (negative control) and oral treatment of free insulin (100 IU/kg) (positive control). As expected, none of the groups showed any significant reduction of blood glucose level (p<0.05) at any point in time during the 4 hour study.

FIG. 14 shows data from 3 different treatment groups of diabetic rats which were orally given insulin Aspart-loaded particles of BaSO₄, BaSO₃, and BaCO₃. Baseline blood glucose level was read before the treatment. All 3 insulin-loaded Ba salt formulations showed reduction in blood glucose level within 1 hour of oral administration.

For insulin-loaded BaSO₄ particles, blood glucose level was significantly low compared to baseline level in 1 to 3 hours (p<0.05). At the 4^{th} hour, the level was still low compared to the baseline level but not significant, with an ascending trend.

Insulin-loaded BaCO₃ particles showed a significant decrease of blood glucose level at 1 to 2 hours, although blood glucose level started to show a slight rise at the 3^{rd} hour, and by the 4^{th} hour it was back to the baseline level.

Insulin-loaded BaSO₃ particles showed a reduction in blood glucose levels but the reduction was not significant throughout the 4 hour period.

The P value was calculated by applying one-way ANOVA to confirm any significant reduction of blood glucose level compared to pre-treatment base line blood glucose level, at any point in time after treatment.

Effect of Pre-Dosing with Bicarbonate Before Oral Administration of Insulin-Loaded BaSO₄, BaSO₂ and BaCO₃ Particles on Hyperglycaemia

200 mg of bicarbonate was orally administered to each treatment group of diabetic rats followed by oral gavage of insulin-loaded BaSO₄, BaSO₃ and BaCO₃ particles, as above. FIG. 15 shows the effect on blood glucose levels of pre-administering bicarbonate before oral gavage of insulin-loaded BaSO₄, BaSO₃ or BaCO₃ particles, over a 4-hour period.

FIG. 16 shows the effect on blood glucose levels of oral administration (a) only with insulin, (b) only with insulin-loaded BaSO₃ particles, and (c) with bicarbonate followed by insulin-loaded BaSO₄ particles.

FIG. 16 clearly shows that the best protocol for reducing blood glucose levels with oral insulin-loaded BaSO₄ particles is with pre-treatment oral administration of bicarbonate.

### Example 6: Albumin Loading Efficiency of BaSO₄, BaSO₃ and BaCO₃ Particles and Subsequent Release of Albumin from the Particles at Acidic pH

The capability of the BaSO₄, BaSO₃ and BaCO₃ particles in protecting protein molecules was further assessed using albumin protein against different pHs and enzymatic effect.

Different amounts of albumin bound to the BaSO₄, BaSO₃ and BaCO₃ particles were separated from the unbound proteins by centrifugation and ran on SDS-PAGE to see the band intensity of the bound proteins following Coomassie dye staining (FIG. 17). The particles were synthesized in the presence of albumin at pH 7.8. A few assays were done with albumin-loaded particles following exposure of the particles to harsh acidic pH of 1.8. All of the BaSO₄, BaSO₃ and BaCO₃ particles were prepared in the presence of 1000 μg/ml, 800 μg/ml and 500 μg/ml of albumin at pH 7.8 and the band intensity for the loaded albumin was the same. The albumin band for concentration of 100 μg/ml initially added to prepare the albumin-loaded particles showed slightly lower intensity. That indicates that albumin at 500 μg/ml might saturate the (albumin) binding sites of the particle.

At acidic pH, albumin loaded into BaSO₄ particles showed slightly decreased intensity compared to the synthesis pH of 7.8 with clear and intact band. That indicates that the BaSO₄ particle can protect albumin from enzymatic degradation even at very harsh acidic pH. However, albumin loaded to BaSO₃ and BaCO₃ particles showed significant degradation at harsh acidic pH. For both BaSO₃ and BaCO₃ particles, an intact but very low intensity band was found for the highest concentration of 1000 μg/ml of albumin. Bands in lower concentrations of albumin were either too dim in intensity or invisible, indicating a significant amount of protein loss even in particle bound form.

### Example 9: Protection of Albumin-Loaded BaSOM BaSO₃ and BaCO₃ Particles from Enzymatic (Trypsin) Digestion

To assess the potential ability of BaSO₄, BaSO₃ and BaCO₃ particles to protect particle-bound proteins from enzymatic digestion in the gastrointestinal tract, albumin-loaded BaSO₄, BaSO₃ and BaCO₃ particles were exposed to trypLE (trypsin mimicking enzyme)-added medium prepared with different pHs (pH 7.4, 6.8 and 1.8). Free undigested and trypsin-digested albumin samples were taken as control.

The trypsin-digested free albumin showed partial fragmentation, whereas albumin proteins loaded into BaSO₄ and BaSO₃ particles remained intact at all 3 different pHs (FIG. 18). BaCO₂ particle-bound albumin was found intact in alkaline pH (7.8 and 6.8); however, the protein band was not visible at acidic pH of 1.8 (FIG. 18).

FIG. 19 shows comparative graphs with relative percentage of protein degradation for different BaSO₄, BaSO₃ and BaCO₃ particles. The band intensity was measured using ImageJ. Relative degradation percentage was calculated from the difference in band intensity between trypLE-digested albumin at pH 7.4 and trypLE-digested albumin at subsequent pHs.

### Example 10: Protection of Albumin-Loaded BaSO₄, BaSO₃ and BaCO₃ Particles in Simulated Gastric Fluid (sGF)

Albumin-loaded BaSO₄, BaSO₃ and BaCO₃ particles were exposed to simulated gastric fluid (sGF). sGF was prepared with added pepsin and in 3 different pHs 5.0, 2.5 and 1, mimicking the fluctuating pH environment of the stomach. The effect observed on band intensity actually reflects the combined effect of enzymatic activity of pepsin and pH, FIG. 20A shows albumin bands from free albumin without any treatment, followed by 3 rows with free albumin exposed to sGF. No visible band for albumin was found when free albumin was exposed to sGF, whereas the 3 visible bands on the gel were from pepsin itself.

Albumin when loaded into the BaSO₄ particle and exposed to sGF with different pHs showed a reduction in band intensity with more acidic pH. Both albumin and pepsin bands were visible at pH 5.0 and pH 2.5. While pepsin band intensity remained the same, albumin band intensity seemed to be dimmed at pH 2.5 compared to pH 5. There was no visible band for albumin or pepsin at pH 1.0.

As shown in FIG. 20B, albumin loaded into BaSO₃ particles and subsequently exposed to sGF with pH 5.0 gave a clear bend, whereas loaded albumin exposed to sGF with pH 2.5 showed a decrease in band intensity, and no albumin band was visible at pH 1.0.

FIG. 20C demonstrated that the band intensity of the albumin loaded into BaCO₃ particles remained the same when exposed to sGF with pH 5 and pH 2.5, while no visible band was found at pH 1.0.

The above results suggest that unlike free albumin, salt nanoparticle-bound albumin could better tolerate extreme acidic pH. The lack of visibility of albumin band at pH 1 could be due to overexposure of the insulin-loaded nanoparticles to the extreme acidic pH or non-release of insulin from undegraded particles.

### Example 11: Effect of Oral Administration of Insulin-Bound SrSO₃ on Blood Glucose Levels

This example illustrates the effect of orally administered insulin-loaded SrSO₃ nano-precipitate on blood glucose levels.

SrSO₃-insulin conjugation was prepared by adding 100 μl of SrCl₂ (1M), 200 IU/kg of insulin and 40 μl or Na₂SO₃ as per the protocol of Examples 1 and 2. After a 14-hour starvation, 200 mg of bicarbonate was administered followed by oral gavage of SrSO₃-insulin conjugation. FIG. 21 shows the effect on blood glucose levels over a 6-hour period.

The above was repeated for SrSO₃-insulin conjugation with 100 IU/kg of insulin and the same treatment protocol was applied after a 14-hour starvation, FIG. 22 shows the effect on blood glucose levels over a 6-hour period.

The above was repeated for SrSO₃-insulin conjugation with 50 IU/kg of insulin and the same treatment protocol was applied after a 14-hour starvation. FIG. 23 shows the effect on blood glucose levels over a 6-hour period.

The above was repeated for SrSO₃-insulin conjugation with 100 IU/kg of insulin. 200 mg of sodium bicarbonate was orally administered three times at 10-minute intervals without starvation followed by oral administration of SrSCh-insulin conjugation with 100 IU/kg of insulin. FIG. 24 shows the effect on blood glucose levels over a 6-hour period.

The above was repeated for SrSO₃-insulin conjugation with 100 IU/kg of insulin, which was orally administered without sodium bicarbonate, after 14-hour starvation, FIG. 25 shows the effect on blood glucose levels over a 6-hour period.

As a negative control, free insulin (5 IU/kg) was intravenously administered. FIG. 26 shows the effect on blood glucose levels over a 6-hour period.

As could be seen from FIGS. 21 to 25, there was a significant decrease in blood glucose levels in the beginning. In particular, from FIG. 23, it was demonstrated that only a very low concentration of insulin (5 IU/kg) bound to SrSO₃ could exert an efficacious effect on reduction of blood glucose levels.

Also pertinent, is the comparison between FIGS. 22 and 25 i.e. administration of oral bicarbonate before oral gavage with the SrSO₃-insulin conjugation versus non-administration of bicarbonate before oral gavage with the SrSO₃-insulin conjugation.

It is clear from FIGS. 22 and 25 that oral administration of bicarbonate before oral treatment with insulin-loaded nano-precipitates of this invention significantly increased the percentage of reduction of blood glucose levels in the test subjects.

### Example 12: Effect of Transferrin and Casen Surface Modification of Insulin-Loaded BaSO₄ on Blood Glucose Levels

This example illustrates the effect of transferrin and casein surface modification of Insulin-loaded BaSO₄ precipitates on blood glucose levels.

BaSO₄-insulin loaded precipitates were prepared by adding 100 μl of BaCl₂(1M), 100 IU/kg of insulin and 40 μl of Na₂SO₄, as per the protocol of Examples 1 and 2. The insulin-loaded precipitates were surface modified with addition of 250 μl (10 mg/ml) of transferrin. The transferrin-modified BaSO₄-insulin loaded precipitates were incubated at 37° C. for 10 minutes before administration to the rats by oral gavage. FIG. 27 shows the effect on blood glucose levels over a 6-hour period following oral administration. Surface modification of the BaSO₄ particles with transferrin helped sustained reduction of blood glucose levels, compared to unmodified BaSO₄ particles. This could be due to the effect of transferrin in facilitating blood absorption of the insulin-loaded nanoparticles across the intestinal epithelium.

For casein-modification, BaSO₄-insulin loaded precipitates were prepared by adding 100 μl of BaC (1M), 100 IU/kg of insulin and 40 μl of Na₂SO₄, as per the protocol of Examples 1 and 2. The insulin-loaded precipitates were surface modified with addition of 250 μl from 1 mg/ml of casein solution. The casein-modified BaSO₄-insulin loaded precipitates were incubated at 37° C. for 15 minutes and then topped up to a total volume of 500 μl with milliQH2O before being administered to the rats by oral gavage. FIGS. 28 and 29 show the effect on blood glucose levels and the percentage of reduction in blood glucose over a 4-hour period following oral administration. The result suggests that casein, when adsorbed on the surface of the insulin-loaded nanoparticles, could play a positive role in further reducing blood glucose levels, probably by facilitating intestinal transport and blood absorption of the insulin-loaded nanoparticles.

### CONCLUSION

For efficacious oral delivery of protein therapeutics such as insulin, it is necessary to attach the insulin molecule to a “carrier” that carries it safely through the gastrointestinal tract. The “carrier” has to meet the criteria for an oral delivery candidate i.e. able to resist the harsh environment of the gastrointestinal tract and enable the insulin molecules to be absorbed through the intestinal wall. The gastrointestinal tract is a large and complex organ system where each different chamber has its own pH and enzymatic environment. Orally administrated protein therapeutics often show poor bioavailability due to the physical as well as physiological barriers encountered in the gastrointestinal tract. One of the biggest challenges is protein degradation by the extreme acidic pH of the stomach. The second major issue is the enzymatic degradation that occurs in both the stomach and intestine. Even after successfully bypassing the stomach pH and enzymatic action, the protein-loaded particles or the released proteins need to overcome the mucin barrier of the gastrointestinal lining, prior to crossing the epithelium either via transcellular or paracellular route to reach blood circulation.

The particle stability study carried out in Example 4 was to assess the ability of the salt nano-precipitates of this invention to withstand the harsh pH conditions of the gastrointestinal tract, specifically the stomach. Successful oral delivery can only take place if the salt nano-precipitates are resistant even in the face of the fluctuating pH environment of the stomach. Another important factor in oral delivery of therapeutics is stomach and intestinal residence time. Depending on the particle properties, the residence time inside the gut could vary from 20 minutes to 3 hours (Gao Y. et. al. (2017)²³). An ideal oral delivery carrier must be resistant to both basic and acidic pHs within that timeframe. The stomach has fluctuating pH, which can vary from pH 1.7 to 4.7, whereas intestinal pH is around 6 to 8 (Koziolek M et. al. (2015)²⁴).

Turbidity, which was measured as absorbance at 320 nm increases as particle formation is accelerated and decreases as particle formation is inhibited or particle dissolution takes place. The BaSO₃, BaSO₃ and BaCO₃ precipitates were prepared at pH 7.8 and then exposed to lower pHs. The results showed that BaSO₄ particles had the highest synthesis rate at pH 7.8 as well as the best resistance at all pHs throughout 3 hours, followed by the BaCO₃ and BaSO₃ particles. All of the BaSO₄, BaSO₃ and BaCO₃ are suitable as oral delivery carriers based on pH resistance.

Presence of the cation-providing and anion-providing salts in the nano-precipitate at a sufficiently high concentration to enable adequate saturation of cationic and anionic salts, per volume of the nano-precipitate, is key to strong binding of the target protein molecule and buffering against premature release at an acidic pH.

In Example 5, fluorometric assay was performed with a fixed amount of FITC-insulin added prior to formation of insulin-loaded particles to assess the insulin loading efficiency of BaSO₄, BaSO₃ and BaCO₃ particles and subsequent release of insulin from the particles at lower pHs. All of the BaSO₄, BaSO₃ and BaCO₃ particles showed very good insulin loading efficiency (80 to 100%). The insulin-loaded particles were also found to be stable as the pH was gradually reduced from 7.78 to 5.0, with almost no release of insulin from the complexes. At pH<5, different degrees of insulin release were observed. Insulin release from BaSO₄ particles was only at 30% even at very harsh acidic pH, implying that this particular nano-insulin formulation would be stable inside the stomach regardless of whether the subject is fed or in a fasting state. Insulin-loaded BaSO₃ and BaCO₃ particles showed almost 80 to 100% release in acidic pH of nearly 1.0.

Mucin of the gastrointestinal lining is the primary barrier for any molecule to cross the intestinal lining and reach systemic circulation. Orally delivered insulin-loaded nano-precipitates of this invention will be in contact with mucin while crossing the intestinal lining.

The adhesion of insulin-loaded BaSO₄, BaSO₃ and BaCO₃ particles to mucin was assessed with FT-IR and Bradford protein assay kit in Example 6 to predict whether the particles would be capable of crossing the intestinal lining. The FT-IR bands showed characteristic peaks for the BaSO₄, BaSO₃ and BaCO₃ particles and mucin, and the protein assay quantitatively confirmed mucin adhesion to the particles. All of the BaSO₄, BaSO₃ and BaCO₃ particles were found to have high mucin adhesion (60 to 100%).

The effect of orally administrated insulin-loaded nano-precipitates of this invention on hyperglycaemia were assessed in Example 7. All of the insulin-loaded BaSO₄, BaSO₃ and BaCO₃ particles resulted in a significant reduction of blood glucose level. It was surprisingly observed that the effect generated by oral administration of the insulin-loaded BaSO₄, BaSO₃ and BaCO₃ particles was similar to the effect of subcutaneous delivery of commercial human insulin aspart (Novorapid, Novonordisk) i.e. lasting 4 to 5 hours with a maximum drop in blood glucose level during the 2 to 3 hour mark. For the orally administered insulin-loaded BaSO₄, BaSO₃ and BaCO₃ particles, onset of action on hyperglycemia started at the 1-hour mark. This could be due to the time required for the insulin-loaded particles to transport the loaded or released insulin into the blood stream. In particular, the BaSO₄ and BaCO₃ particles were found to dramatically reduce hyperglycaemia, working in the same way as subcutaneously administered insulin aspart in terms of onset and duration of action. The percentage of reduction of blood glucose level in comparison to the baseline level showed a maximum reduction below 50% at any point in time for all BaSO₄, BaSO₃ and BaCO₃ particles.

The results of Example 11 suggests that SrSO₃ particles could also help in protecting insulin from degradation by acidic pH and hydrolytic enzymes in the gastrointestinal (GIT) tract while enabling blood absorption of insulin across the intestinal epithelium.

As will be readily apparent to those skilled in the art, the present invention may easily be produced in other specific forms without departing from its scope or essential characteristics. The present embodiments are, therefore, to be considered as merely illustrative and not restrictive, the scope of the invention being indicated by the claims rather than the foregoing description, and all changes which come within therefore intended to be embraced therein.

### REFERENCES

¹Curto, M. D. D. et. al. (2011): Oral Delivery System for Two-pulse Colonic Release of Protein. Drugs and Protease Inhibitor/Absorption Enhancer Compounds. J. Pharm. Sci. 2011,100, 8.
²Zaman, R et. al. (2016): Carrier Mediated Systemic Delivery of Protein and Peptide Therapeutics. Curr. Pharm. Des. 2016, 22(999),1-1.
³Allen, C. et. al. (2011): Digestive system structure and function. In Roesch, ed. Laboratory Manual for Anatomy and Physiology, 4th ed. 2011. New Jersey: John Wiley & Sons, Inc. pp. 570,
⁴Banga, A. K, (2006): Oral Delivery of Peptide and Protein Drugs. In Therapeutic Peptides and Proteins Formulation, Processing, and Delivery Systems. 2^{nd} ed; Boca Raton: CRC Press, Taylor & Francis Group, 2006, pp. 229-50.
⁵Mathiowitz, E. et. al. (1997): Biologically erodable microspheres as potential oral drug delivery systems. Nature. 1997, 386, 410-4.
⁶Ensign, L. M. et. al. (2012): Oral drug delivery with polymeric nanoparticles: The gastrointestinal mucus barriers, Adv.Drug.Deliv, Rev. 2012, 64, 557-70.
⁷Wu, Z, M, et. al. (2012): HP55-coated capsule containing PLGA/RS nanoparticles for oral delivery of insulin. Int.J.Pharm. 2012, 425, 1-8.
₈Pan, Y, et. al. (2002): Bioadhesive polysaccharide in protein delivery system: chitosan nanoparticles improve the intestinal absorption of insulin in vivo. Int. J. Pharm. 2002, 249, 139-47.
⁹Krauland, A. H. et. al. (2004): Oral insulin delivery: the potential of thiolated chitosan-insulin tablets on nondiabetic rats. J. Control, Release. 2004; 95(3): 547-55.
¹⁰Moiero, J. E. et. al. (1982): Orally administered liposome entrapped insulin in diabetic animals. Horm. Res. 1982, 16, 249-56,
¹¹Stefanov, A. V. et. al. (1980): Effect of liposomally entrapped insulin administered per os on the blood sugar level in normal and experimentally diabetic rats. Ukr. Biokhim. Zh. 1980, 52, 497.
¹²Haiberg, I. et. al. (2018): Efficacy and safety of oral basal insulin versus subcutaneous insulin glargine in type 2 diabetes: a randomised, double-blind, phase 2 trial. Lancet, Diabetes, Endocrinol. 2018, 18, 30372-3
¹³Drucker, D. 1 (2019): Advances in oral peptide therapeutics. Nat. Rev. Drug. Discov. 2019. 19, 277-289.
¹⁴Gregory, J. M. et. al. (2019): Enterically delivered insulin tregopil exhibits rapid absorption characteristics and a pharmacodynamic effect similar to human insulin in conscious dogs. Diabetes Obes Metab. 2019, 21, 160-169,
¹⁵Arbit, E et, al. (2017): Oral insulin delivery in a physiologic context: review. J. Diabetes Sci. Technol. 2017, 11, 825-832.
¹⁶Geho, W. B. et. al. (2014): A single-blind, placebo-controlled, dose-ranging trial or oral hepatic-directed vesicle insulin add-on to oral antidiabetic treatment in patients with type 2 diabetes mellitus. J. Diabetes Sci. Technol. 2014, 8, 551-559.
¹⁷Ahmad, A et. al. (2015): Controlled release of insulin in blood from strontium-substituted carbonate apatite complexes. Curr.D.Deliv. 2015, 12(2), 210-22.
¹⁸Sifontes, Ä. B. et. al. (2015): Obtaining Highly Crystalline Barium Sulphate Nanoparticles via Chemical Precipitation and Quenching in Absence of Polymer Stabilizers. J. Nanomater, 2015, 2015, 1-7.
¹⁹Ramaswamy, V. et. al. (2010): Synthesis and characterization of BaSO₄ nano-particles using micro emulsion technique. Adv. Appl. Sci. Res. 2010, 1(3), 197 204.
²⁰Qiao, X. et. al. (2018): Controllable synthesis of nanostructured BaSO₄ and BaSO₃ crystals on the basis of DMSO oxidation chemistry. Cryst. Eng. Comm. 2018, 20, 173.
²¹Sreedhar, B. et. al. (2012): Shape Controlled Synthesis of Barium Carbonate Microdusters and Nanocrystallites using Natural Polysachharide—Gum Acacia. Am. J. Mater. Sci. 2012, 2, 2(1):5-13.
²²Liu, F. et. al. (1988): Influence of water on the Infrared Spectra of Mucin. Mikrochim. Acta. 1988, I, 357-359.
²²Gao, Y. et. al, (2017): Oral Formulations for Preclinical Studies: Principle, Design, and Development Considerations (Chapter 17). In Developing Solid Oral Dosage Forms. 2nd Ed. Ed.; Qiu Y, Zhang G.G.Z. Chen Y; Mantri R.V. Pharmaceutical Theory and Practice. 2017. pp. 455-495.
²⁴Koziolek, M. et. al. (2015): Investigation of pH and Temperature Profiles in the GI Tract of Fasted Human Subjects Using the Intellicap® System. JPDDR. 2015, 104, 2855-2863.

1. An oral insulin delivery salt nano-precipitate, said salt nanoprecipitate comprising:
an inorganic cation-providing metal salt for electrostatic binding with the negative charges of insulin, said cation-providing salt being a barium salt (Ba²⁺); and
an anion-providing salt for electrostatic binding with the positive charge of insulin, said anion-providing salt being a sulphate (SO₄²⁻);
wherein both the cation-providing and anion-providing salts are present in the nano-precipitate at a sufficiently high concentration to enable adequate saturation of cationic and anionic salts, per volume of the nano-precipitate, such that the insulin is strongly bound therewith and buffered against premature release at an acidic pH; characterized in that,
said insulin-loaded salt nano-precipitate is surface modified with a bio-adhesive protein selected from either transferrin or casein.

2. The salt nano-precipitate according to claim 1, wherein the ratio of said cation-providing inorganic metal salt to said anion-providing salt is 5:2.

3. The salt nano-precipitate according to claim 1, wherein said salt nano-precipitate is barium sulphate (BaSO₄).

4. A method of producing the salt nano-precipitate of claim 1, said method comprising the following steps:
(i) providing a volume of said cation-providing inorganic metal salt;
(ii) adding a volume of insulin to the cationic metal salt solution of step (i);
(iii) providing a volume of said anion-providing salt and adding the same to the mixture of step (ii);
(iv) mixing the constituents of the resulting mixture of step (iii) until a precipitate is formed; and
(v) adding a volume of said bio-adhesive protein to the precipitate of step (iv).

5. The method according to claim 4, wherein said cation-providing inorganic metal salt is barium chloride (BaCl₂).

6. The method according to claim 4, wherein said anion-providing salt is sodium sulphate (Na₂SO₄).

7. The salt nano-precipitate of claim 1 for use in the treatment of hyperglycemia, type 1 or type 2 diabetes mellitus in an animal or a human subject.

8. The use of claim 7, wherein bicarbonate is orally administered before treatment with the insulin-loaded salt nano-precipitates.
